(19) United States
(12) Patent Application Publication
(10) Pub. No.: US 2025/0331771 A1
(43) Pub. Date: Oct. 30, 2025

(54) SYSTEM AND METHOD OF MODELING ERYTHROPOIESIS

(71) Applicant: Fresenius Medical Care Deutschland, GMBH, Bad Homburg (DE)

(72) Inventors: David Josef Jorg, Frankfurt (DE); Doris Helene Fuertinger, Long Island City, NY (US)

(73) Assignee: Fresenius Medical Care Deutschland, GMBH, Bad Homburg (DE)

(21) Appl. No.: 19/180,707

(22) Filed: Apr. 16, 2025

Related U.S. Application Data
(60) Provisional application No. 63/639,211, filed on Apr. 26, 2024.

(51) Int. Cl. A61B 5/00; A61B 5/145; A61B 5/1455; A61M 1/14; G16B 5/00; G16H 10/60; G16H 20/10

(52) U.S. Cl. CPC A61B 5/4839; A61B 5/14535; A61B 5/1455; G16B 5/00; G16H 10/60; G16H 20/10; A61M 1/14

(57) ABSTRACT

Methods and systems are disclosed for assisting with the management of anemia in a patient. In some examples, the method includes accessing patient parameters associated with the patient, wherein the patient parameters include an aligned hemoglobin time series for the patient generated from multiple measurement sources. The method then includes accessing a physiology-based model and adapting the physiology-based model into a patient specific model that predicts future hemoglobin levels for the patient based on one or more erythropoiesis-stimulating agent (ESA) dosing regimens, wherein adapting the physiology-based model to the patient specific model utilizes the patient parameters, and generates estimates of patient-specific physiological characteristics. The method then includes running simulations with the patient specific model and determining a recommended ESA dose. The recommended ESA dose that the model predicts will cause either or both of the patient's hematocrit or hemoglobin concentration to reach a desired range within a specified time frame.

### CROSS REFERENCE TO RELATED APPLICATIONS

This application claims the benefit of U.S. Provisional Patent Application No. 63/639,211, filed Apr. 26, 2024, and entitled “System and Method of Modeling Erythropoiesis,” the entirety of which is incorporated by reference herein.

### FIELD OF DISCLOSURE

The present disclosure relates to modeling cell production. More particularly, the present disclosure relates to systems and methods of modeling erythropoiesis.

### BACKGROUND

Red blood cells (erythrocytes) are essential for the transport of oxygen through the body. An understanding of the regulation of red blood cell production, called erythropoiesis, is important for the treatment of patients in a variety of clinical situations. Patients that are scheduled for elective surgery, such as hip or transplant surgery, can be prescribed an erythropoiesis stimulating agent (ESA) to compensate for the expected loss of blood, thus obviating the need for allogenic blood transfusions by raising the patient's hematocrit and/or hemoglobin concentration to a desired range at the predetermined time, in expectation of the surgery.

ESAs, including recombinant human erythropoietin, exert hematological effects analogous to the hormone erythropoietin (EPO), which is released into the blood stream by the kidneys based on a negative feedback mechanism that reacts to the partial pressure of oxygen in the blood. ESA treatment regimens are also prescribed for patients who suffer from insufficient erythropoiesis, such as cancer patients recovering from the effects of chemotherapy, and chronic kidney disease patients whose kidneys can no longer produce sufficient amounts of EPO. The dose and frequency of administration of an ESA treatment regimen are often determined based on the prior experience of the physician and on established guidelines, because predictive models of erythropoiesis under an ESA treatment regimen are not readily available.

Therefore, there is a need for a predictive model of erythropoiesis under various ESA treatment regimens.

### SUMMARY

The present disclosure describes models for erythropoiesis in humans based on structured population models for the different cell stages in development, from stem cells in bone marrow to erythrocytes in the blood stream. The models may be configured to predict a patient's hematotocrit and/or hemoglobin concentration resulting from a treatment regimen. A non-limiting example of a treatment regimen may include an erythropoiesis stimulating agent (ESA) regimen.

The models may be or may include personalized models configured for individual patients based on parameters or clinical data associated with the patient. The personalized models may receive patient parameters as input and generate a predicted hematotocrit and/or hemoglobin concentration as output. The patient parameters may include various information including, without limitation, gender, height, recent body weights, hemoglobin levels, hematotocrit, ESA doses, and/or the like.

In some embodiments, the hemoglobin level patient parameter may be determined using a hemoglobin time series process. For example, the patient parameters may use clinical data that includes laboratory hemoglobin measurements and/or hematocrit data (for instance, measured using a non-invasive device). Patient hemoglobin levels can exhibit considerable fluctuation. Accordingly, the hemoglobin time series process provides an approach for developing a hemoglobin time series for a patient utilizing both the laboratory hemoglobin and the hematocrit data that addresses and overcomes issues with patient hemoglobin fluctuation. For example, the hemoglobin time series process operates to smooth patients' hemoglobin levels utilizing both the hemoglobin and the hematocrit data for use with the models described according to various embodiments.

In one embodiment, a method of adjusting a patient's hematocrit and/or hemoglobin concentration to a desired range at a predetermined time with an erythropoiesis stimulating agent (ESA) regimen includes obtaining patient parameters required for input into a model for predicting the patient's hematocrit and/or hemoglobin concentration at a predetermined time with a selected ESA administration regimen, and employing the patient parameters and an initially selected EPO administration regimen in the model to predict the patient's hematocrit and/or hemoglobin concentration at the predetermined time with the initially selected ESA administration regimen. Optionally, if the patient's hematocrit and/or hemoglobin concentration is not predicted by the model to be in the desired range at the predetermined time, the method includes employing the model with one or more different ESA administration regimens until the model predicts that the patient's hematocrit and/or hemoglobin concentration will be in the desired range at the predetermined time. The method then includes administering ESA to the patient with the ESA administration regimen predicted to adjust the patient's hematocrit and/or hemoglobin concentration to the desired range at the predetermined time. The patient parameters can include the starting hematocrit and/or hemoglobin concentration in the patient's blood, the total blood volume of the patient, the lifespan of red blood cells (RBCs) of the patient, the mean corpuscular volume of the RBCs, and the rate of neocytolysis in the patient's blood.

The predetermined time can be, for example, in a range of between about 5 days and about 200 days into the ESA administration regimen. In some embodiments, the patient undergoes a medical procedure prior, during, or after initiation of an ESA administration regimen, including medical procedures such as blood donation, surgery, and dialysis, or any combination thereof. For dialysis patients, the desired hematocrit can be, for example, in the range of between about 28 percent and about 36 percent and the desired hemoglobin concentration can be, for example, in a range of between about 9.5 g/dL and about 12 g/dL.

In yet another embodiment, a computer system for adjusting a patient's hematocrit and/or hemoglobin concentration to a desired range at a predetermined time with an erythropoiesis stimulating agent (ESA) regimen includes a user input means for determining patient parameters from a user, including, in some embodiments, patient hemoglobin levels determined via a hemoglobin time series process, a digital processor coupled to receive determined patient data from the input means, wherein the digital processor executes a modeling system in working memory, and an output means coupled to the digital processor, the output means provides to the user the patient's hematocrit and/or hemoglobin concentration under the ESA administration regimen at the predetermined time. The modeling system employs the patient parameters and an initially selected EPO administration regimen in the model to predict the patient's hematocrit and/or hemoglobin concentration at the predetermined time with the initially selected ESA administration regimen. Optionally, if the patient's hematocrit and/or hemoglobin concentration is not predicted by the model to be in the desired range at the predetermined time, employs the model with one or more different ESA administration regimens until the model predicts that the patient's hematocrit and/or hemoglobin concentration will be in the desired range at the predetermined time.

In still another embodiment, a method of determining a patient's hematocrit and/or hemoglobin concentration within a desired range at a predetermined time with an erythropoiesis stimulating agent (ESA) regimen includes obtaining patient parameters, including, in some embodiments, patient hemoglobin levels determined via a hemoglobin time series process, required for input into a model for predicting the patient's hematocrit and/or hemoglobin concentration at a predetermined time with a selected ESA administration regimen, and employing patient parameters and an initially selected EPO administration regimen in the model to predict the patient's hematocrit and/or hemoglobin concentration at the predetermined time with the initially selected ESA administration regimen. Optionally, if the patient's hematocrit and/or hemoglobin concentration is not predicted by the model to be in the desired range at the predetermined time, the method includes employing the model with one or more different ESA administration regimens until the model predicts that the patient's hematocrit and/or hemoglobin concentration will be in the desired range at the predetermined time. The method then can include administering ESA to the patient with the ESA administration regimen predicted to adjust the patient's hematocrit and/or hemoglobin concentration to the desired range at the predetermined time.

Processes and parameters according to some embodiments have many technological advantages over existing computer-based erythropoiesis modeling and/or anemia treatment systems. One non-limiting technological advantage may include the achievement of an ESA regimen needed for a desired hematocrit and/or hemoglobin concentration for a patient, thereby, on the one hand, alleviating insufficient erythropoiesis, and, on the other hand, preventing excessively high ESA dose levels that raise the patient's blood pressure and increase the patient's risk of stroke and cardiovascular disease. Another non-limiting technological advantage may include providing smooth, non-fluctuating data patient hemoglobin and/or hematocrit data to the models to improve the accuracy of model predictions. Additional non-limiting technological advantages would be understood by those of skill in the art in view of the present disclosure.

The embodiments described herein further include a method for generating an aligned hemoglobin time series for a patient from multiple measurement sources. Moreover, a method of assisting with the management of anemia in a patient is provided.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments of the present disclosure.

FIG. 1 is a schematic illustration of an anemia therapy assistance system, according to some embodiments.

FIG. 2 is an example diagram depicting the process of erythropoiesis, according to some embodiments.

FIG. 3 sets forth model equations, according to some embodiments.

FIG. 4A is a list of definitions for model parameters for the equations set forth in FIG. 3, according to some embodiments.

FIG. 4B is a list of model parameters for the equations set forth in FIG. 3 considering a shortened transit time of progenitor cells, according to some embodiments.

FIG. 5 depicts experimental results of models and hemoglobin time series processes, according to some embodiments.

FIGS. 6A-6C depict experimental results of models and hemoglobin time series processes, according to some embodiments.

FIG. 7 depicts experimental results of models and hemoglobin time series processes, according to some embodiments.

FIG. 8 is a schematic view of a computer network in which the described embodiments may be implemented, according to some embodiments.

FIG. 9 is a block diagram of an example computer system, according to some embodiments.

FIG. 10 is a block diagram of an example computing environment, according to some embodiments.

FIG. 11 is a block diagram of an example computing environment, according to some embodiments.

FIG. 12 is a flow chart illustrating various steps in an example method, according to some embodiments.

FIG. 13 is a flow chart illustrating various steps in an example method, according to some embodiments.

### DETAILED DESCRIPTION

Red blood cells (erythrocytes) are essential for the distribution of oxygen through the body to organs and tissues. They take up oxygen in the lungs and deliver it to tissues while squeezing through the capillaries. To fulfill this task properly, they are highly specialized. For instance, being shaped like biconcave disks optimizes the oxygen exchange. Furthermore, they give up their nuclei, organelles, and mitochondria in order to provide more space for hemoglobin, the molecule which oxygen binds to. Erythrocytes are very deformable and can therefore pass capillaries half their diameter. During microcirculation, they have to withstand high shear stresses, rapid elongation, folding, and deformation. Over time, the cell membrane is damaged by these extraordinary stresses. Because of the lack of nuclei and organelles, red blood cells cannot divide or repair their cell membranes. Senescent erythrocytes lose their flexibility due to their fragmented membranes. These stiff cells could do harm to small capillaries or even clog them. To avoid this potential harm, old erythrocytes are recognized by phagocytes and destroyed. This phagocytosis mainly takes place in the spleen and cells of the reticulo-endothelial system (RES).

To compensate for phagocytosis of senescent red blood cells, it is necessary to build new erythrocytes continuously. The maturation of undifferentiated stem cells to mature erythrocytes is called erythropoiesis and takes place in the bone marrow. Erythropoiesis not only has to compensate for the continuous loss of old erythrocytes, but also for the additional loss of cells due to random breakdown, as well as due to internal and external bleeding. Furthermore, the number of red blood cells has to be adjusted to varying environmental conditions, as for instance a transition from low to high altitudes or vice versa, by increasing the rate of erythropoiesis, or, conversely, by neocytolysis, a process believed to be wherein macrophages start to phagocytose young erythrocytes (neocytes).

During the process of erythropoiesis, the cell population undergoes a series of proliferations and differentiations. Starting from multipotential stem cells, erythroid cells mature to BFU-Es (earliest stage of erythroid committed cells), CFU-Es, different stages of erythroblasts, and finally reticulocytes. The reticulocytes are released from the bone marrow into blood and mature within 1-2 days to erythrocytes.

The primary control of erythropoiesis is governed by the hormone erythropoietin (EPO). EPO is released into the blood stream by the kidneys based on a negative feedback mechanism that reacts to the partial pressure of oxygen in blood. The concentration of EPO affects the number of circulating red blood cells by determining the number of cells that mature into erythrocytes, either by recruitment or by preventing apoptosis (programmed cell death), and by affecting the velocity of maturing of progenitor and precursor cells. Thus, disturbances in oxygen delivery can be adjusted for by an adaptive resetting of the rate of erythropoiesis. Additionally, as already mentioned above, there exists a physiological process which affects the selective degradation of young erythrocytes in situations of red cell excess, called neocytolysis. Neocytolysis seems to be triggered by a drop in the EPO level.

Another critical factor for effective erythropoiesis is the availability of iron which is indispensable for hemoglobin synthesis. If the body is not able to provide sufficient iron for this process, then ineffective erythropoiesis will result In normal subjects, the total iron content of the body stays within narrow limits (iron overload is toxic). Once an atom of iron enters the body it is conserved with remarkable efficiency and can remain in the body for more than ten years. Iron is lost via loss of cells (especially epithelial cells), bleeding and loss of very small amounts via urine and sweat. The balance of iron content is achieved by absorption and not by control of excretion. If the plasma concentration of iron is too low, then the level of the hormone hepcidin is decreased. The consequence of a lower hepcidin level is that more iron is taken up via the duodenum and more iron is released from macrophages and from the stores. Patients suffering from inflammation, such as dialysis patients, typically have higher hepcidin levels. Increasing iron availability in inflamed dialysis patients can be achieved by an increase of parenteral iron by increasing dose, frequency, or both, and by reducing inflammation by diagnosis and treatment of sources of inflammation, e.g., barrier breakdown (i.e., skin, periodontal disease, intestinal congestion), pulmonary or urinary tract infection, thrombosed fistulas or catheter, and by subsequent specific therapy, e.g., antibiotics, catheter removal, aseptic techniques when manipulating in-dwelling catheters, and surgical debridement of skin ulcers.

Since individual cells in the various cell populations which have to be considered have to be distinguished according to their age, age-structured population models are needed in order to describe the development of the cell populations. Besides these age-structured population models, the model of various embodiments may include a feedback loop including erythropoietin. In the model development below, iron supply is fixed to a rate which corresponds to a sufficient supply of iron one would expect in a healthy person (without iron deficiency).

Maintaining stable hemoglobin levels within predefined target levels using existing systems, including existing computer-and/or machine learning-based systems, can be challenging, particularly in patients with frequent hemoglobin fluctuations both above and below the desired targets. Personalized dosing of erythropoiesis stimulating agents (ESA) can improve hemoglobin target attainment.

Anemia is common in patients with chronic kidney disease (CKD), particularly in those receiving dialytic kidney replacement therapy, such as hemodialysis. Per the 2022 USRDS Annual Data Report (“Clinical Indicators and Preventive Care”), 78.2% of hemodialysis patients received erythropoiesis-stimulating agents (ESAs). Further, among this patient population, methoxy polyethylene glycol-epoetin beta use increased from 24.1% in 2016 to 39.3% in 2020.

A hemoglobin target range of about 10 to about 11 g/dl or about 10 to about 11.5 g/dL is suggested by clinical guidelines such as Kidney Disease Outcomes Quality Initiative (KDOQI) (2007), Kidney Disease Improving Global Outcomes (KDIGO) (2012) and U.S. Food and Drug Administration (FDA) (2011) recommendations. Achieving and maintaining target hemoglobin levels is challenging due to various factors, including biological heterogeneity, a nonlinear dose-response relationship, and time delays between hemoglobin measurement, subsequent ESA administration, and hemoglobin response. In hemodialysis patients, hemoglobin levels frequently vary above and below set targets within short time intervals even if mean hemoglobin levels remain within the target range.

Various tools have been introduced over the years to support physicians with anemia management, including computer-and machine learning-based software tools. Treatment protocols are frequently used and considered standard of care in many dialysis clinics. Additionally, software tools, many of which apply machine learning techniques, have been developed and implemented in clinical practice. However, existing computer-and machine learning-based software tools have many shortcomings, including, without limitation, inaccurate hemoglobin concentration predictions and lack of interpretability of the underlying decision-making process that prevents the clinician from understanding individual ESA dose recommendations.

Accordingly, some embodiments provide an anemia therapy assistance system that comprises comprehensive physiology-based models of erythropoiesis and erythrocyte dynamics that estimates patient-specific key physiological characteristics. For each patient, the system creates a set of personalized models utilizing clinical data (e.g., gender, height, recent body weights, hemoglobin levels, and ESA doses). Through the use of specific patient's data according to some embodiments, these general models may be personalized, essentially creating a patient's “digital twin” or “patient avatar.” A model predictive controller processes the models' predictions of individual hemoglobin trajectories to provide fully personalized ESA dosing recommendations. The models are specifically configured to achieve and maintain hemoglobin levels within narrow target ranges using ESA efficiently.

The clinical data used as patient input for the models may include hemoglobin and hematocrit measurements. Patient hemoglobin levels can exhibit considerable fluctuation and the amount of hemoglobin measurements (e.g., time resolution) may be less than desired. Accordingly, some embodiments may use a hemoglobin time series process for developing a hemoglobin time series for a patient utilizing both the laboratory hemoglobin and the hematocrit data that smooths out fluctuations and provides improved accuracy and time resolution. These embodiments provide an improvement over existing systems which do not account for those fluctuations and have deficient time resolution, and therefore, the ESA dosage to patients is not as effective, is too low, or is too high. As such, generating the hemoglobin time series for the patient improves existing techniques and provides a recommendation for ESA dosage that is more efficient and more effective.

Moreover, the proposed systems and methods depict an improvement to processing efficiency of predictive modeling for ESA dosages. For example, predictive models that predict ESA dosages to assist in treating anemia can require substantial data that requires considerable processing resources and less efficacious dose recommendations require more time and iterations before achieving a desired outcome. However, by generating the smoothed hemoglobin time series, with improved accuracy and time resolution as described herein, and then feeding the smoothed hemoglobin time series into the predictive modeling, this improves the efficiency and accuracy of the predictive modeling. The smoothed time series as described herein aides in improving the efficiency and speed of the predictive model, providing a more efficacious ESA dosage recommendations in a shorter time frame with fewer iterations than existing systems.

In one embodiment, a method of adjusting a patient's hematocrit and/or hemoglobin concentration to a desired range at a predetermined time with an ESA regimen includes obtaining patient parameters required for input into a model for predicting the patient's hematocrit and/or hemoglobin concentration at a predetermined time with a selected ESA administration regimen, and employing the patient parameters and an initially selected EPO administration regimen in the model to predict the patient's hematocrit and/or hemoglobin concentration at the predetermined time with the initially selected ESA administration regimen. Examples of ESAs are provided in Table 1 (adapted from Phurrough S, Jacques L, Ciccanti M, Turner T, Koller E, Feinglass S: “Proposed Coverage Decision Memorandum for the Use of Erythropoiesis Stimulating Agents in Cancer and Related Neoplastic Conditions”; Centers for Medicare and Medicaid Services; Administrative File: CAG #000383N; May 14, 2007). Note that unlike other ESAs listed in Table 1, Peginesatide is not a biologically derived EPO, it is a synthetic peptide that stimulates EPO receptors.

TABLE 1
Erythropoiesis Stimulating Agents: EPO = erythropoietin.

| Compound | Drug Names | Manufacturer |
|---|---|---|
| EPO α | Epogen ® | Amgen |
| EPO α | Procrit ® | Amgen |
| EPO α (w/o serum | Eprex ® Epypo ® | J&J subsidiary |
| albumin) | Epopen ® Epoxitin ® | (Otho Biologics) |
| | Globuren ® | |
| EPO β | (Neo)Recormon | Roche |
| EPO β | Erantin ® | Boehringer |
| | | Mannheim (Spain), |
| | | Roche (Spain) |
| EPO β | Epoch ® | Chugai |
| EPO δ in human | Dynepo Gene | Aventis |
| cell lines | Activated EPO | Transkaryotic |
| | | Therapies |
| EPO Ω | Epomax ® Hemax ® | Baxter |
| | Hemax ®-Eritron ® | |
| Modified EPO α | Aranesp ® | Amgen |
| Darbepoietin | | |
| Modified EPO α | Nespo ® | Amgen |
| Darbepoietin | | |
| Modified EPO β | Mircera ® | Roche |
| Continuous EPO | | |
| Receptor Activator | | |
| (Pegylation) | | |
| Peginesatide | Omontys ® | Affymax |

Optionally, if the patient's hematocrit and/or hemoglobin concentration is not predicted by the model to be in the desired range at the predetermined time with the initially selected ESA administration regimen, the method includes employing the model with one or more different ESA administration regimens until the model predicts that the patient's hematocrit and/or hemoglobin concentration will be in the desired range at the predetermined time. The method then includes administering ESA to the patient with an ESA administration regimen predicted to adjust the patient's hematocrit and/or hemoglobin concentration to a value within the desired range at the predetermined time. The patient parameters can include the starting hematocrit and/or hemoglobin concentration in the patient's blood, the total blood volume of the patient, the lifespan of red blood cells (RBCs) of the patient, the mean corpuscular volume of the RBCs, and the rate of neocytolysis in the patient's blood.

In some embodiments, the hematocrit and/or hemoglobin concentration in the patient's blood used as parameter input to models according to various embodiments can be obtained from routine laboratory measurements known in the art. In various embodiments, the hematocrit and/or hemoglobin concentration in the patient's blood used as parameter input to models according to various embodiments can be calculated using hemoglobin time series processes according to various embodiments.

In some embodiments, the total blood volume (BV) of the patient can be estimated as described further below, or measured by use of radio-labeling red blood cells with chromium-51 to estimate red blood cell volume (RCV) and using the formula

$$BV = RCV / ( 0.9 * Hctv )$$

where Hctv is the venous hematocrit, obtained from routine laboratory measurements known in the art.

In some embodiments, devices, including non-invasive devices, may be used to measure certain properties of patient blood, for instance, during dialysis by taking measurements of blood flowing through the extracorporeal circuit of a dialysis system. For example, the Crit-Line® Monitor (CLM), available from Fresenius Medical Care Waltham, Massachusetts, United States of America, may measure patient hematocrit (which may be used to determine hemoglobin (Hgb) levels) and/or relative blood volume (RBV) information during dialysis. In another example, the CliC device available from Fresenius Medical Care, Waltham, Massachusetts, United States of America may measure absolute hematocrit, RBV, and continuous oxygen saturation.

The hemoglobin concentration may be determined via hemoglobin time series processes according to various embodiments.

The lifespan of RBCs of the patient can be estimated from endogenous alveolar carbon monoxide concentrations. The mean corpuscular volume can be obtained from routine laboratory measurements known in the art. The rate of neocytolysis in the patient's blood can be estimated from correlations with reduced expression of CD44 (homing-associated cell adhesion molecule) and CD71 (transferrin receptor).

Models according to various embodiments may be configured to track the patient's predicted hematocrit and/or hemoglobin concentration over time, such as between about 5 days and about 200 days of the ESA administration regimen. The predetermined time can be any future time after an ESA administration regimen is selected and the predicted regimen is initiated. In some embodiments, the patient undergoes a medical procedure prior, during, or after the ESA administration regimen, such as blood donation, surgery, and dialysis, or any combination thereof. For dialysis patients, the desired hematocrit is typically in the range of between about 28 percent and about 36 percent and the desired hemoglobin concentration is typically in a range of between about 9.5 g/dL and about 12 g/dL. For elective orthopaedic surgery patients, the desired hemoglobin concentration for males and females is typically greater than or equal to 13 g/dL, and 12 g/dL, respectively.

In another embodiment, a method of determining a patient's hematocrit and/or hemoglobin concentration within a desired range at a predetermined time with an erythropoiesis stimulating agent (ESA) regimen includes obtaining patient parameters required for input into a model for predicting the patient's hematocrit and/or hemoglobin concentration at a predetermined time with a selected ESA administration regimen, and employing the patient parameters and an initially selected EPO administration regimen in the model to predict the patient's hematocrit and/or hemoglobin concentration at the predetermined time with the initially selected ESA administration regimen. Optionally, if the patient's hematocrit and/or hemoglobin concentration is not predicted by the model to be in the desired range at the predetermined time, or a different ESA administration regimen is desired due to other considerations, the method includes employing the model with one or more different ESA administration regimens until the model predicts that the patient's hematocrit and/or hemoglobin concentration will be in the desired range at the predetermined time. The method then can include administering ESA to the patient with the ESA administration regimen predicted to adjust the patient's hematocrit and/or hemoglobin concentration to a value within the desired range at the predetermined time.

Models according to some embodiments include a comprehensive physiology-based model of erythropoiesis and erythrocyte dynamics that estimates patient-specific key physiological characteristics, such as red blood cell lifespan. For each patient the system creates a set of personalized models utilizing routine clinical data (gender, height, recent body weights, hemoglobin levels, and ESA doses). A model predictive controller processes the models' predictions of individual hemoglobin trajectories to provide fully personalized ESA dosing recommendations. The software is specifically designed to achieve and maintain hemoglobin levels within narrow target ranges using ESA efficiently.

Models according to some embodiments may be the same or similar to models described in U.S. Pat. No. 10,319,478, titled “System and Method of Modeling Erythropoiesis and its Management,” and/or U.S. Pat. No. 9,679,111, titled “System and Method of Modeling Erythropoiesis Including Iron Homeostasis,” both of which are incorporated by reference in the present disclosure.

The models may be used as part of an anemia therapy assistance system operative to model, calculate, predict, or otherwise determine an ESA does to attain a target hemoglobin concentration (e.g., at or about 10.5 g/dl, the mid-point of the target range of about 10.0 g/dl to about 11.0 g/dl). The system can support health care professionals (HCP) to manage anemia in hemodialysis patients. The system may operate to compute individualized ESA dose recommendations at certain time intervals, for instance, every fourteen days.

### ANEMIA THERAPY ASSISTANCE SYSTEM

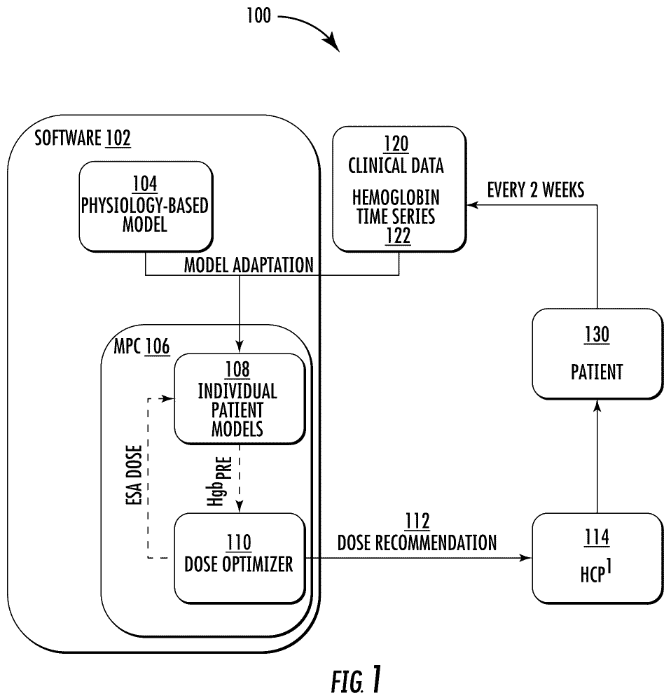

FIG. 1 depicts an illustrative anemia therapy assistance system 100 according to some embodiments. In general, system 100 may be configured to generate hemoglobin concentration predictions for ESA dosage regimens using models configured according to some embodiments. The hemoglobin concentration predictions can be used to generate treatment recommendations, for instance, ESA dosages that lead to a predicted hemoglobin concentration within a target range.

As shown in FIG. 1, system 100 may include a computer or software system 102 configured to execute various computer-implemented models. In some embodiments, the models may include a physiology-based model of erythropoiesis 104. In various embodiments, the physiology-based model 104 may operate according to embodiments described in the present disclosure. For example, the physiology-based model 104 may comprehensively simulate or otherwise model the production of erythrocytes, spanning from stem cells committing to the erythroid lineage, burst-forming unit cells, colony-forming unit cells, erythroblasts and bone marrow reticulocytes to the red cells circulating in the blood stream. The physiology-based model 104 may mathematically describes the proliferation, apoptosis and differentiation of the various cell types and the influence of ESAs and endogenous erythropoietin on these processes. In some embodiments, the physiology-based model 104 may also include neocytolysis, a phenomenon recognized to contribute to renal anemia. In some embodiments, the physiology-based model 104 may model the pharmacokinetics of the endogenous erythropoietin and exogenous ESAs as described with a pharmacokinetic model.

In various embodiments, software system 102 may include a patient personalization module 108 configured to adapt the physiology-based model 104 to individualized patients 130. For example, in various embodiments, demographic data (gender, height, pre-and/or post-hemodialysis weight, and/or the like) may be used to estimate the euvolemic blood volume using the Nadler formula (for instance, the The Nadler and Allen formula described below) and to calibrate the number of stem cells committing to the erythroid lineage. In some embodiments, a global optimization strategy estimates physiological key characteristics of anemia from recent clinical data of the patient 130 (hemoglobin levels and ESA doses). These biological key characteristics may include the red blood cell life span, endogenous erythropoietin production, ESA half-life, ESA dependent apoptosis rate of erythrocyte progenitor cells, the ESA dependent maturation function of erythrocyte precursor cells, and/or the like. Individualized patient models may be configured to satisfy a set of quality criteria, for instance, that the mean absolute percentage error between hemoglobin data and model output (i.e. simulated hemoglobin values) is less than a threshold value (e.g., 5.5%).

A model predictive controller (MPC) 106 may be configured to calculate the next recommended ESA doses. In general, several possible physiological states (i.e. sets of model parameters) are compatible with the patient's 130 retrospective clinical data 120. Hence, one or more individualized models 108 may be used to describe a patient instead of only a single model. A multi-stage model predictive control with a robust horizon strategy for the next recommended dose 112 may be used to incorporate the information of multiple models. An open-loop problem may be formulated and repeatedly solved for the set of personalized models for an upcoming duration (e.g., sixteen weeks). The MPC 106 may be configured to determine a single ESA dose for the next recommended dose 112 for all considered patient models such that the predicted hemoglobin curves stabilize within the hemoglobin target range over a duration, for instance, the next eight weeks. In general, only the next recommended dose is unambiguous. Subsequent doses may vary between different patient models. In some embodiments, predictions may be evaluated for their quality, for instance, defined by the difference in the predictions of hemoglobin outcomes and differences in dosing strategies between the different patient models and the area outside the target range.

A healthcare provider (HCP) 114 may use the recommended dose 112 to provide or recommend a dose or dose regimen for the patient 130.

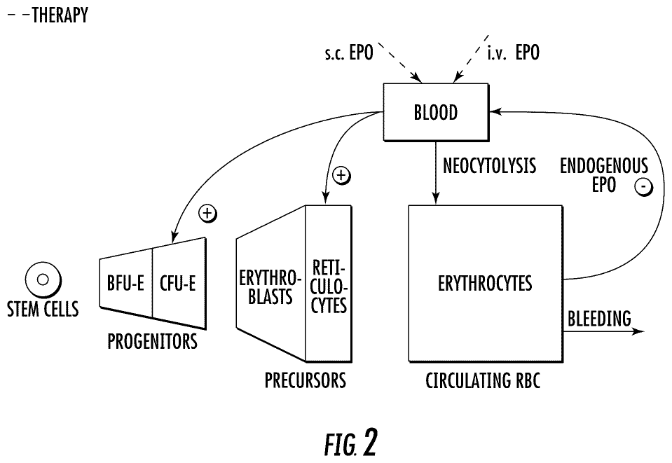

In various embodiments, the clinical data (or patient parameters) 120 provided to the software system 102 for use with the models may include a hemoglobin time series 122 determined using a hemoglobin time series process according to some embodiments. FIG. 2 illustrates an example process of erythropoiesis.

The clinical data collected to evaluate a patient's blood hemoglobin level may include measured hemoglobin and hematocrit data. Hemoglobin measurements assess the concentration of hemoglobin in patient blood, whereas hematocrit measures the proportion of red blood cells in the blood, which may be measured via a non-invasive device described below (e.g., Crit-Line® Monitor (CLM) or CliC device). Those measurements are related with each other but not necessarily interchangeable. Further the hemoglobin (or laboratory hemoglobin) measurement may be done on a blood sample of the patient, whereas a non-invasive device, in one example, may use photo-optical technology to arrive at its measurement. In dialysis patients blood draws are typically done at the beginning or the end of the treatment and non-invasive measurements are continuously taken/taken with a specified frequency throughout the treatment. Over the course of a dialysis treatment, typically, fluid is removed from the vascular space, for instance, the blood volume changes throughout the treatment. This impacts measured hemoglobin concentrations as well as the proportion of red blood cells in the blood. To use both data sources simultaneously to assess the anemia status/hemoglobin concentration of the patient the measurements need to be aligned with each other and systematic biases minimized (e.g. due to different time points when measurements are taken in the course of the treatment).

One application for a combined laboratory hemoglobin and non-invasive hematocrit measurements time series can be to assess hemoglobin fluctuations in the patient over time, for instance, over the course of several weeks or months. Patient hemoglobin levels can exhibit considerable fluctuations. The techniques according to some embodiments can employ a novel approach to smoothing of the hemoglobin levels utilizing both the hemoglobin and the hematocrit data. This smoothing approach can be utilized for all patients and/or selectively utilized for patient's that may be classified as having hemoglobin levels that are “fluctuating.” This smoothing approach may additionally be used to select patients in need for better control of their anemia and who consequently might profit from the use of a personalized dosing system, such as the anemia ITAS.

A patient's hemoglobin time series may be classified as “fluctuating” within a specified time window, for example, if one and/or both of following two criteria are fulfilled:
1. The hemoglobin difference between the minimum and the maximum of the time series is larger than 1.75 g/dL.
2. The fraction of time that the weekly hemoglobin rate of change exceeds 0.1 g/dL/week is larger than 60%.

This fluctuating time series classification may be evaluated on the raw clinical data 120 or a smoothed hemoglobin time series.

### Hemoglobin Time Series Process

In some embodiments, a hemoglobin time series may be or may include a combined laboratory hemoglobin and hematocrit time series, where the hematocrit measurements are derived from non-invasive continuous blood monitoring during the treatment. Laboratory hemoglobin measurements are used where available and hematocrit measurements otherwise. Hemoglobin values are computed from hematocrit (Hct) measurements according to one or more of the following:
Both Hct (in units of %) at the start and the end of the dialysis treatment are lowpass-filtered, for instance, removing all data points that lie further than a threshold (e.g., 5) percentage points from a moving average computed in a window +/−a time period (e.g., 10 days) around the respective data point.
Laboratory hemoglobin (in units of g/dl) is lowpass-filtered, removing all data points that lie further than a threshold (e.g., 5/3 g/dl) from a moving average computed in a time period window (e.g., +/−15 days) around the respective data point.
Hct values at the end of treatment are ignored if there was an unexpected treatment end at the day of the measurement.
A time series containing days (or another time period) and measurements where hemoglobin from all three sources (laboratory and continuous monitoring measurements) are present is created.
The following steps are carried out for a moving time window (e.g., 150 days) length starting from the beginning of this time series:
   Both Hct values (in units of %) are divided by a concentration percentage value (e.g., 3%/(g/dl)) to convert to hemoglobin (in units of g/dl), (“pre-and post-treatment hemoglobin”).
   The mean offset between the pre-treatment hemoglobin and the laboratory hemoglobin values are computed and subtracted from the pre-and post-treatment hemoglobin to align its mean with laboratory hemoglobin.
A single interpolation factor between the pre-and post-treatment hemoglobin is computed such that the average deviation between the resulting interpolated hemoglobin and lab hemoglobin is minimized. Both the resulting pre-and post-treatment hemoglobin and laboratory hemoglobin are combined to a single time series using lab hemoglobin values on days where it is available and pre-and post-treatment hemoglobin values otherwise.
The resulting time series may be required to contain a minimum number of data points (at least 15 data points) and/or stretch over a minimum time (e.g. 90 days) for the computation of the fluctuating status.
A spline interpolation of the time series may be generated. A non-limiting example of generating the time series may include using Python via the SciPy function scipy.interpolate.splrep with smoothing parameter s=0.18*(number of hemoglobin data points). Both the resulting hemoglobin spline and its first derivative are evaluated on a time grid with a spacing value (e.g., 0.1 days).

For criteria testing:
Criterion #1 is fulfilled if the difference between the maximum and the minimum of the hemoglobin spline interpolation is greater than a threshold value (e.g., 1.75 g/dL).
Criterion #2 is fulfilled if the number of time grid points for which the first derivative of the hemoglobin spline interpolation is larger than a threshold value (e.g., 0.1g/dL/week) and is more than a threshold percentage (e.g., 60%) of the total number of time grid points of the interpolated time series.

The combined hemoglobin time series (e.g., lab hemoglobin values and hematocrit-based measurements) may be utilized by the anemia therapy assistance system 100 when calculating the ESA dose 112.

### ERYTHROPOIESIS AND ITS REGULATORY MECHANISMS

The daily production rate in a healthy adult totals about 200×10⁹ red blood cells under normal conditions. This number can vary due to varying internal and environmental conditions. The body has to be able to adapt the number of erythrocytes to situations of anemia and/or hypoxia (e.g. after bleeding, due to a shortened RBC lifesp an, due to an enhanced eryptosis, . . . ) as well as to situations of excessive red blood cells (e.g., high altitude dwellers descending to sea level).

The production of new erythrocytes takes place in the bone marrow. Undifferentiated stem cells in the bone marrow commit to the erythroid lineage and undergo a series of proliferations and differentiations. The earliest stage of erythroid committed cells are called BFU-Es (Burst-Forming Unit Erythroid). Within a few days the BFU-Es mature to CFU-Es (Colony-Forming Unit Erythroid), then they undergo different stages of erythroblasts and finally they become reticulocytes. The reticulocytes are released to the blood stream and within 1-2 days they appear as mature erythrocytes. Throughout the description below, the term progenitor cells is used to refer to BFU-E and CFU-E cells and the term precursor cells is used to subsume proerythroblasts, basophilic erythroblasts, orthochromatophilic erythroblasts and bone marrow reticulocytes.

The primary control of erythropoiesis is governed by the hormone erythropoietin. EPO is produced mainly by peritubular cells in the renal cortex based on a negative feedback mechanism. The kidneys detect the partial pressure of oxygen in blood and react by releasing more EPO if the oxygen content is too low and vice versa. The concentration of EPO affects the number of circulating RBC by determining the number of cells that mature into erythrocytes. Erythropoietin plays a role in the recruitment of stem cells to the erythroid lineage, prevents apoptosis of progenitor cells and affects the maturation velocity of progenitor and precursor cells. Thus, disturbances in oxygen delivery can be adjusted for by an adaptive resetting of the rate of erythropoiesis, as shown in FIG. 1. Additionally, there exists a physiological process, wherein macrophages start to phagocytose young erythrocytes (neocytes), which is called neocytolysis. Neocytolysis seems to be triggered by a drop in the

EPO level and helps to regulate situations of excessive red cell mass. (See Rice 2001). Further, recent studies suggest that the concentration of EPO in blood influences the clearance of senescent RBCs and that EPO can prolong the lifespan of erythrocytes by inhibiting cation channels.

### Progenitor and Precursor Cells

Once a stem cell committed to the erythroid lineage, the cell can not regress or switch to another hematopoietic lineage, but can only develop to an erythrocyte. The number of stem cells which enter the different hematopoietic lineages is determined by interleukines and growth factors. The immature erythrocyte undergoes a number of changes in structure, appearance and its requirements during the process of differentiation and proliferation. BFU-E cells, the first committed erythroid cells, express only a very small number of EPO-receptors (EpoR) and therefore they are almost EPO-independent for their survival. Within a few days they develop into CFU-E cells.

As cells mature to CFU-Es the number of EpoR on the cell surface increases distinctly and the cells become absolutely dependent on erythropoietin to prevent them from apoptosis. Under normal conditions, large numbers of generated CFU-E are not surviving. After that very EPO-sensitive phase the density of EpoRs declines sharply on early erythroblasts and EpoRs almost disappear at the stage of orthochromatophilic erythroblasts. Under high levels of EPO the marrow transit time of precursor cells is shortened and high EPO levels result in release of still immature reticulocytes, which are referred to as stress reticulocytes.

One significant difference between progenitor and precursor cells is the synthesis of hemoglobin. Unlike progenitor cells, all types of precursor cells synthesize heme and the increased demand for iron for this process is reflected by a sharp increase in the number of transferrin receptors (TfR) in proerythroblasts (transferring receptors are the only path of iron uptake). TfRs reach their peak in intermediate erythroblasts and decline afterward and mature bone marrow reticulocytes express only a few TfRs (in comparison).

### Neocytolysis

The body has to be able to deal with situations of too few circulating erythrocytes as well as with excessive red blood cells. Whereas it is very effective to change the rate of erythropoiesis to adapt the system to situations when red cell numbers are low, this is not an adequate regulatory mechanism in situations of red cell mass excess. This is because a decrease in the rate of erythropoiesis takes relatively long to have a noticeable effect on the number of circulating red blood cells, due to the long lifespan of erythrocytes.

Previously, preventing apoptosis of red cell progenitors had been thought to be the sole regulating effect of EPO. Because this would only allow for a slow adaption in situations of too many RBC, the control of the body of red cell mass would be coarse. A decline in erythropoietin level results in more progenitor cells dying but a suppression of the hormone has no effect on maturation of erythroid precursors. Thus, no decrement in the erythrocyte production would be observable within one week after EPO has declined. However, studies done on residents at very high altitude who rapidly descended to sea level, showed a decrease in red cell mass of 10-18% in the first 7-10 days. (See Rice 2001). Persons living in hypoxic environments, like high altitude, are normally polycythemic. Thus, their hematocrit is too high under normal conditions and as a consequence the release of EPO is suppressed.

Further investigations of situations where EPO levels are lower than normal (polycythemic high altitude dwellers, anemia of renal failure, human model based on EPO administration then withdrawal), suggest that a suppression of EPO leads to selective hemolysis of young red blood cells. This process is called neocytolysis, to stress the fact that young erythrocytes (=neocytes) are uniquely susceptible.

Neocytolysis is initiated by a fall in erythropoietin levels and in Rice 2005 it was shown that, for instance, low doses of EPO administered to high altitude dwellers on descent, completely abrogated neocytolysis. At the moment it remains unclear whether it is the rate of decline in EPO level or the drop of EPO beneath a certain threshold that acts as a trigger for neocytolysis.

The current approach to treatment of anemia with ESAs deviates significantly from the normal internal systemic process. Neocytolysis, for instance, contributes to renal anemia. It is precipitated by the pathologic endogenous erythropoietin deficiency of renal disease and the short bursts in EPO concentration followed by sharp declines in serum levels due to administration of the hormone (especially during intravenous (i.v.) administration). It would be desirable to choose dosing schedules such that neocytolysis is minimized or totally prevented. Models according to various embodiments may be configured to better define administration schemes which stimulate erythropoiesis in a more natural way and abrogate neocytolysis.

### Iron and Erythropoiesis

Erythropoiesis is a very complex process and even though erythropoietin is the key regulator, there are other proteins (e.g., interleukins, . . . ) and substances (e.g., iron, folic acid, vitamin B₁₂, . . . ) that are needed for an optimal erythropoiesis. For instance, one very critical factor for an effective production of red blood cells is iron. The metal is indispensable for hemoglobin synthesis and the hemoglobin protein is the actual oxygen transporter in erythrocytes. The molecule makes up about 97% of the dry weight of red blood cells. If the body is not able to provide sufficient iron for the production of erythrocytes, then impaired erythropoiesis will result. In general, a healthy adult has difficulty providing sufficient iron to support production rates greater than three times basal. Higher rates may be possible when administering EPO and iron and in some diseases, e.g. hemochromatosis. An undersupply of iron additionally leads to an increase in the number of hypochromic RBC. Hypochromic cells (i.e., cells in which the amount of hemoglobin is lower than the normal 26 pg) are small, have a reduced oxygen carrying capacity and are relatively fragile, and thus are likely to die earlier than normochromic erythrocytes.

Erythroid precursor cells are the most avid consumers of iron in the body. The immature erythrocyte has only a few days time to synthesize all the hemoglobin which the mature cell contains. Hemoglobin is a metalloprotein. Its name refers to the special structure. A hemoglobin molecule consists of four subunits each composed of a globular protein embedding a heme group, and every heme group in turn contains one iron atom. Erythroid cells rely completely on transferrin receptors to take up iron. Contrary to progenitor cells, all types of precursor cells synthesize heme and the increased demand for iron for this process is reflected by a sharp increase in the number of TfR in proerythroblasts. Transferrin receptors reach their peak in intermediate erythroblasts followed by a decrease with further maturation.

In precursor cells heme is essential for maintaining a “normal” number of TfR. Studies showed that inhibition of heme synthesis strongly inhibited TfR expression. It seems that there exists a positive feedback mechanism in which heme promotes a high rate of transferrin receptor synthesis. A high number of transferrin receptors enhances iron uptake and that in turn keeps hemoglobin synthesis at high levels. (P. Ponka. Tissue-specific regulation of iron metabolism and heme synthesis:

Distinct control mechanisms in erythroid cells. There is evidence that precursor cells which have a low hemoglobin content are still able to proliferate but do not differentiate and undergo apoptosis.

### Anemia of Renal Disease

Anemia affects almost all patients with chronic kidney disease. It is caused by failure of renal excretory and endocrine function. It often develops once renal function decreases to 50% and the degree of anemia increases with severity of renal failure. Anemia develops because of deficiency in endogenous erythropoietin production by the kidneys (note that even non functioning kidneys produce some EPO and can maintain hemoglobin levels higher than those found in anephric patients), increased blood losses (gastrointestinal bleeding (purpura, gastrointestinal and gynecologic bleeding occur in one third to one half of all CKD patients), frequent blood sampling, blood left in the dialyzer, multiple vascular access surgeries, . . . ), functional or absolute iron deficiency and decreased red cell survival. Furthermore, neocytolysis contributes to renal anemia and it explains the often demonstrable hemolytic component and the worsening of hemolysis with more pronounced renal disease. A description of neocytolysis is provided above. It further explains the responsiveness of hemolysis to ESA therapy and the increased efficiency of subcutaneous compared to intravenous therapy, because in s.c. administration nadirs in EPO levels which precipitate neocytolysis are avoided. In general, renal anemia is normocytic and normochromic and the number of reticulocytes is normal or slightly decreased, which is inappropriate in the context of a reduced RBC population. Certain deficiency states, especially iron, but also folate or vitamin B₁₂ deficiency may alter the nature of the anemia.

Untreated anemia is associated with decreased oxygen delivery to the tissues. For compensatory reasons, cardiac output increases, resulting in left ventricular hypertrophy. Cardiac disease is the most common cause of death among patients who are on maintenance dialysis. Partial correction of anemia in these patients was shown to reduce cardiac ischemia and ameliorate cardiomegaly, thus, reducing cardiac related morbidity. Further consequences of uncorrected anemia are decreased cognition and mental acuity and overall decrease in patient welfare.

### ESA Therapy

Erythropoiesis stimulating agents have been used to treat anemia in patients suffering from chronic renal failure for more than two decades. Optimal hemoglobin targets are still a matter of discussion. Studies have shown that a partial correction of anemia is preferable to a full correction. A number of complications can occur in patients with CKD when they have near-normal/normal hemoglobin levels, i.e., higher vascular access thrombosis, hypertension and greater requirements for antihypertensives, cardiovascular events, earlier need for renal replacement therapy and higher mortality. Normal hemoglobin levels for women are in a range of between about 12 g/dl and 16 g/dl, and in a range of between about 13 g/dl and about 17.5 g/dl for men.

Defining an optimal hemoglobin target is not the only issue regarding ESA therapy. Another problem is: how can the target hemoglobin be achieved and how to keep the patient near this hemoglobin level over a long time period? The dose and frequency of administration of an ESA treatment regimen are most often determined based on the prior experience of the physician and on established guidelines. This approach bears some limitations and level of hemoglobin tends to fluctuate greatly and cycling phenomena are observed. An analysis of 31,267 patients on hemodialysis in the Fresenius Medical Care-North America database found that only 5% of patients persistently remained within a desired Hb range of 11-12 g/dl for a period of 6 months. Fishbane et al. ((S. Fishbane and J. S. Berns. Hemoglobin cycling in hemodialysis patients treated with recombinant human erythropoietin. Kidney International, 68:1337-1343, 2005)) analyzed data of dialysis patients collected over five years in a hospital and came to a similar conclusion. More than 90% of the patients experienced hemoglobin cycling. The authors state that changes in ESA dose were the most important driver and were associated with hemoglobin excursion in about 80% of cases. The ESA dose adjustment protocol that was used was similar to the protocol used in most dialysis centers (see Tables A1-A8 of Fishbane for an example of a typical dose adjustment protocol).

Therapy with ESAs is quite different than biological erythropoietin secretion. In hemodialysis patients, i.v. administration route is primarily used, because of the availability of venous access. Intravenous treatment involves short, intermittent, non-physiologic bursts of EPO concentration. The bursts are followed by a fast decline to very low levels of EPO. These fluctuations in plasma concentration do not coincide, either temporally or in magnitude, with physiologic perturbations. Therefore, it may not be surprising that Hb levels fluctuate widely and that it is extremely difficult for physicians to adjust dosing schemes such that no cycling phenomena occur. Note that fluctuations in hemoglobin result in varying oxygen delivery to vital organs. Consequences include repeated episodes of relative ischemia and compensatory mechanisms in organs (e.g. heart) that may result in disordered growth signals, pathologic organ function and worsened patient outcomes. (See Fishbane et al.).

Predictive models of erythropoiesis according to some embodiments can help deal with this situation. For instance, a physician can simulate different ESA treatment regimens and observe their effects on hemoglobin levels over the next few months. Dosing regimens can be tested/chosen with regard to avoidance of neocytolysis, minimal amounts of ESA administered and avoidance of cycling patterns in Hb concentration.

### Iron Therapy

Patients with anemia of chronic kidney disease have to be followed for symptoms of iron deficiency because 80-90% of dialysis patients on ESA therapy will require iron at some stage. This very pronounced need for supplementary iron has different reasons. On one hand iron stores can be depleted (absolute iron deficiency). Iron loss in hemodialysis patients (due to continuous gastrointestinal, purpural and gynecological bleeding, frequent blood sampling, surgeries, etc.) is about 1500-3000 mg/year, as compared to iron loss for a healthy adult that is about 400-800 mg/year, and can be even higher under certain circumstances. Hence, the daily need for iron can be well above the absorptive capacity of the intestinal. This is aggravated by the fact that the uptake via the duodenum is often impaired in these patients. On the other hand, a functional iron deficiency is often observed in renal anemia. During ESA therapy, the number of erythroid progenitor and precursor cells in the bone marrow increase drastically and this imposes a lot of stress on systemic iron homeostasis. Supply and demand often do not match. It is difficult, even for healthy persons, to increase the rate at which iron is released from stores and is recycled from hemoglobin to deliver enough iron to the bone marrow to keep up with supraphysiologic rates of RBC production during ESA treatment. Matters are further complicated in chronic kidney disease because of inflammation, which frequently occurs with various degrees of severity. Thus, iron utilization is regularly decreased in renal insufficiency. See above for a detailed description of the effects of inflammation on systemic iron homeostasis.

In the pre-ESA era iron overload, because of successive blood transfusions, was a major cause of morbidity in dialysis patients. Its significance in the post-EPO era remains unclear. A continuous administration of i.v. iron certainly results in overfilled iron-stores and imposes serious health risks. Hence, decisions on when to supply the patient with i.v. iron and when to withdraw therapy have to be thoroughly evaluated. A mathematical model can help keep track of the current iron status of a patient and can help to make decisions on adaptations of treatment.

Finally, there is a very complex interaction between hemoglobin cycling and iron storage whose dynamical behavior under ESA and iron treatment, even for an experienced physician, is barely predictable. In Alfrey et al., the authors suggest that: “[ . . . ] the current therapeutic paradigm of hemoglobin monitoring, iron treatment, and rHuEPO treatment results in recurrent nonphysiologic cycling of hemoglobin levels in hemodialysis patients.”

### MATHEMATICAL MODEL OF ERYTHROPOIESIS IN AN ADULT

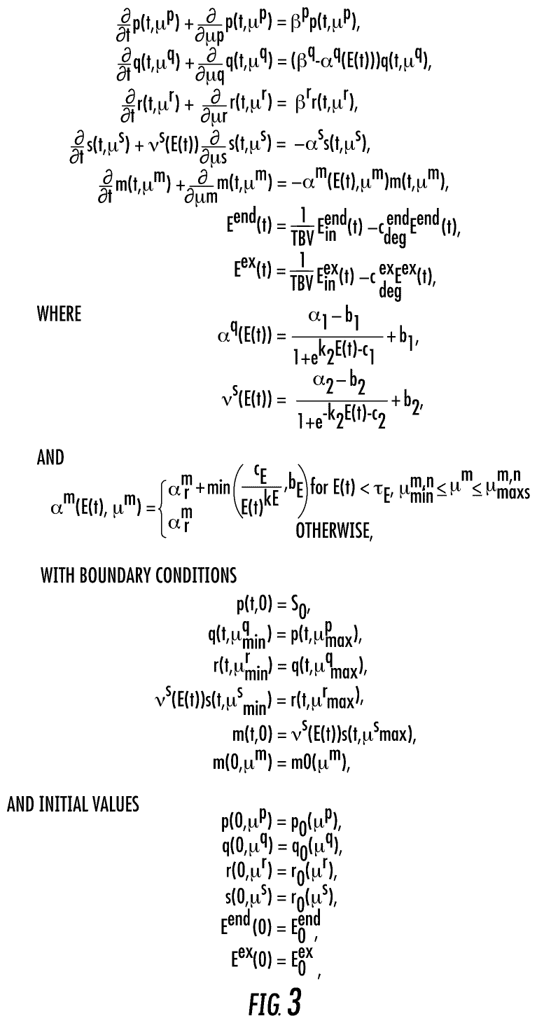

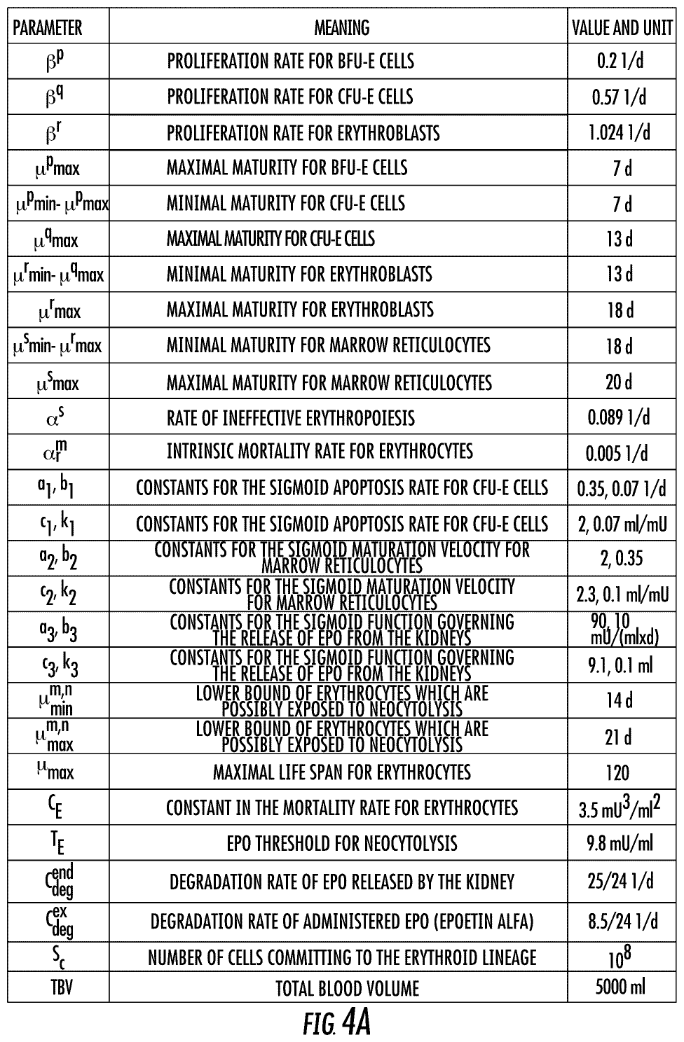

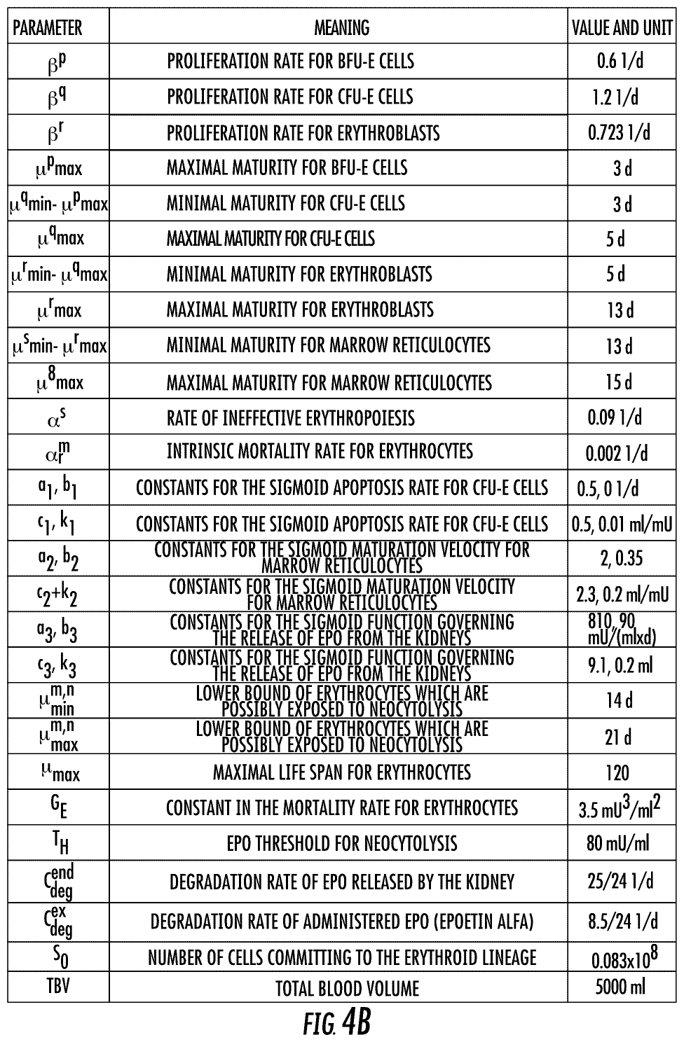

Equations described below are also provided in FIG. 3. Moreover, FIG. 4A provides a list of definitions for model parameters for the equations set forth in FIG. 3. FIG. 4B is a list of model parameters for the equations set forth in FIG. 3 considering a shortened transit time of progenitor cells.

The model developed below focuses on the effects of erythropoietin on erythroid cells. Hence, throughout this development, an impairment of erythropoiesis because of an under supply with iron is ruled out. Despite this limitation, the mathematical models described in the present disclosure are applicable for a number of different situations. For instance, the model presented here is able to describe the recovery of red cell mass after blood donation, the reaction of the body to presurgical administration of ESAs and changes in the number of erythrocytes of polycythemic high altitude dwellers descending to sea level. This model is only applicable for a small subpopulation of dialysis patients. If iron deficiency is treated by administering iron, and therefore erythropoiesis is sufficiently supplied with it, the model can be applied to a larger subgroup of dialysis patients. Furthermore, this model helps to understand the most important dynamics that need to be considered for red blood cell production.

The model is based on structured population models describing the different erythroid cell stages. Five different population classes of cells are considered: BFU-E, CFU-E, erythroblasts, marrow reticulocytes and mature erythrocytes circulating in the bloodstream (including peripheral reticulocytes). Individual cells are distinguished according to their maturity, which can also be referred to as cell age. The commitment to the erythroid lineage is an irreversible event differentiated cell cannot regress or switch into another differentiation pathway. Thus, once a multipotent stem cell is committed to the erythroid lineage, it undergoes the complete series of differentiations until it becomes a mature red blood cell, or it dies eventually during this process. While maturing the cell divides a number of times. Hence, age-structured population models of the form

$$\frac{\partial }{\partial t} u ( t , μ ) + v ( E ( t ) ) \frac{\partial }{\partial μ} u ( t , μ ) = ( β - α ( E ( t ) , μ ) ) u ( t , μ ) ,$$

are used in order to describe the development of the cell populations. Here u(t, ) denotes the population density of the cell population at time t with maturity. Further, β(·) and α(·) describe the proliferation rate and rate of apoptosis, respectively, of the cells. The function α(·) a priori depends on the maturity and the concentration of EPO E(t), respectively, at time t. The function v describes the maturation velocity and depends on the concentration E(t).

For the different population classes, the characteristic properties (proliferation rate, rate of apoptosis and maturation velocity) change depending on erythropoietin, because while maturing the cell changes its morphological characteristics, such as, the number of EPO-and transferrin-receptors expressed on the surface.

In the following sections, the assumptions that were made for the different population classes are listed, briefly described and the mathematical equations that arise in consequence are stated.

### Progenitor Cells: BFU-E and CFU-E Cells

### Assumptions

1. The number of cells, which commit to the erythroid lineage, is constant.
2. Cells normally stay in this stage for 13 days (7 days BFU-E and 6 days CFU-E).
3. EPO has no effect on the number of divisions or the rate of apoptosis of BFU-E.
4. The proliferation rate of CFU-E cells is constant,
5. whereas the rate of apoptosis depends highly on EPO levels.
6. The maturation velocities of BFU-E and CFU-E cells are constant.

The process by which stem cells are recruited into proliferating progenitor population remains unclear. There are several hypotheses including an environmental dependency, that it is a random event, etc. It may be assumed that the number of stem cells entering the erythroid lineage is independent of EPO and thus constant. The change in population of the progenitor cells over time are described considering two different classes of cells: namely BFU-E and CFU-E cells.

The earliest Identifiable erythroid progenitor cell is the Burst-forming Unit Erythroid (BFU-E). At first, these cells express only a very small number of EPO receptors on the surface. (See Wintrobe's Clinical Hematology). Thus, it is reasonable to assume EPO has no effect on proliferation or apoptosis of these cells. (See Wu et al.). In culture it lasts around 6-7 days until human BFU-E have all the functional characteristics of the next cell stage—called CFU-E (Colony-forming Unit Erythroid) (See Williams Hematology) (Assumption 2). Morphologically it is difficult to distinguish between those two types of cells because there are cells in between these two developmental stages which show characteristic properties between BFU-E and CFU-E. Therefore, a distinction is valid but artificial.

The BFU-E cell class is described by the following population Equation (1):

$$\frac{\partial }{\partial t} p ( t , {μ}^{p} ) + \frac{\partial }{\partial {μ}^{p}} p ( t , {μ}^{p} ) = {β}^{p} p ( t , {μ}^{p} ) , \qquad Equation ( 1 )$$

$$p ( t , 0 ) = {S}_{0} ,$$

$$p ( 0 , {μ}^{p} ) = {p}_{0} ( {μ}^{p} ) ,$$

where p(t, μ^{P}) is the population density of the cell class at time t with maturity μ^{P},

$$0 \leq {μ}^{p} \leq {μ}^{{p}_{max}} = 7 , t > 0 .$$

Further, β^{p} is a constant proliferation rate and μ^{P}≡0 (Assumption 3), S₀ describes the number of cells committing to the erythroid lineage (Assumption 1) and p₀(μ^{p}) is the population density at t=0.

Once a cell reaches the maximum age for BFU-E cells, it leaves this population class and enters the CFU-E class. Consequently, there is a continual flux of cells from one population class to the next one. CFU-E are more rapidly dividing cells than BFU-E. During this stage, cells are very sensitive to EPO levels and under normal conditions large numbers of generated CFU-E are not surviving. (See Wintrobe's Clinical Hematology). CFU-E are highly dependent on EPO to prevent them from apoptosis, i.e., the mortality for this population class depends on the EPO concentration. Altogether, the following equations (Equation 2) for the second class are obtained:

$$\frac{\partial }{\partial t} q ( t , {μ}^{q} ) + \frac{\partial }{\partial {μ}^{q}} q ( t , {μ}^{q} ) = ( {β}^{q} - {α}^{q} ( E ( t ) ) ) q ( t , {μ}^{q} ) , \qquad Equation ( 2 )$$

$$q ( t , {μ}_{min}^{q} ) = p ( t , {μ}_{max}^{p} ) ,$$

$$q ( 0 , {μ}^{q} ) = {q}_{0} ( {μ}^{q} ) ,$$

where q(t, μ^{q}) is the population density of the CFU-E class at time t with maturity μ^{q}9, t>0 and

$$7 = {μ}^{{q}_{min}} \leq {μ}^{q} \leq {μ}^{{q}_{max}} = 13 .$$

Further on, β^{q} stands for a constant proliferation rate (Assumption 4), α^{q}(E(t)) denotes the apoptosis rate depending on the EPO-concentration (Assumption 5),

$$q ( t , {μ}^{{q}_{min}} ) = p ( t , {μ}^{{p}_{max}} )$$

describes the number of cells leaving the BFU-E cell stage and entering the CFU-E cell stage and q₀(μ^{q}) is the population density at t=0.

A sigmoid function (e.g., Equation (3)) is used to describe the rate of apoptosis,

$${α}^{q} ( E ( t ) ) = \frac{{a}_{1} - {b}_{1}}{1 + {e}^{{k}_{1} E ( t ) - {c}_{1}}} + {b}_{1} \qquad Equation ( 3 )$$

where E(t) is the EPO concentration at time t and a₁, b₁, c₁ and k₁ are positive constants and a₁>b₁. The function α^{q} monotonically decreases with increasing EPO concentration. Thus, a higher level of EPO causes more cells to survive.

In view of Assumption 6,ν^{p}=ν^{q}≡1, for all t≥0, and thus, the maturity or cell age of progenitor cells, respectively, actually coincides with the age of the cell.

### Precursor Cells: Erythroblasts and Marrow Reticulocytes Assumptions

7. Cells stay in this stage for 6-8 days (5 days erythroblasts and 1-3 days marrow reticulocytes).
8. The class erythroblasts consists of all cell stages from proerythroblast to orthocromatophilic erythroblast.
9. EPO has no effect on the number of divisions or the rate of apoptosis of erythroblasts. The proliferation rate of erythroblasts is assumed to be constant.
10. The maturation velocity of erythroblasts is constant.
11. Reticulocytes mature but do not proliferate.
12. The maturation velocity of reticulocytes depends on EPO.
13. A constant portion of marrow reticulocytes is phagocytosed.

After a CFU-E differentiates to a proerythroblast, it takes about another 6-8 days until the cell is released from the bone marrow into the bloodstream. The various stages of maturation from proerythroblast to orthochromatophilic erythroblast are referred to as erythroblasts. The cells undergo several mitotic divisions until at the stage of orthochromatophilic erythroblast they lose their ability to divide and enter a maturation period. The erythroblastic pyramids appear normal, with no evidence of additional mitotic divisions, when production increases, i.e., it may be assumed that proliferation of erythroblasts to be independent of EPO levels and define it to be constant. See Williams Hematology.

Hence, the erythroblast class can be described by the following equation (e.g., Equation (4)):

$$\frac{\partial }{\partial t} r ( t , {μ}^{r} ) + \frac{\partial }{\partial {μ}^{r}} r ( t , {μ}^{r} ) = {β}^{r} r ( t , {μ}^{r} ) , \qquad Equation ( 4 )$$

$$r ( t , {μ}_{min}^{r} ) = q ( t , {μ}_{max}^{q} ) ,$$

$$r ( 0 , {μ}^{r} ) = {r}_{0} ( {μ}^{r} ) ,$$

where r(t, μ^{r}) is the population density of the erythroblasts at time t with maturity μ^{r}, t>0 and

$$13 = {μ}_{min}^{r} \leq {μ}^{r} \leq {μ}_{max}^{r} = 18.$$

Further, β^{r} is a constant proliferation rate and α^{r}≡0 (Assumption 9), the maturation velocity ν^{r}≡1 (Assumption 10),

$$r ( t , {μ}_{min}^{r} ) = q ( t , {μ}_{max}^{q} )$$

describes the number of cells leaving the CFU-E stage and entering the erythroblasts cell stage and r₀(μ^{r}) is the population density at t=0.

The differentiating process from orthochromatophilic erythroblasts to marrow reticulocytes involves the extrusion of the cell nucleus. Reticulocytes are not capable of cell divisions (Assumption 11), i.e., β^{s}=0. In this model, an impaired erythropoiesis due to iron deficiency may not be accounted for. Still, even when the erythroid cells in the bone marrow are sufficiently supplied with iron, not all precursor cells survive. Some of the reticulocytes die before they are released to the blood stream. This is why Assumption 13 is made.

A raised EPO concentration shortens the marrow transit time of precursor cells. If the EPO level is elevated marrow reticulocytes are released prematurely, which may be accounted for by allowing the maturation velocity of reticulocytes to vary depending on erythropoietin concentration. Thus, although the maximum cell age of marrow reticulocytes is fixed the actual transit time for the cells varies between 1-3 days. Hence, the transit time for precursor cells is between 6-8 days (Assumption 7).

The population equation referring to marrow reticulocytes is (e.g., Equation (5))

$$\frac{\partial }{\partial t} s ( t , {μ}^{s} ) + {v}^{s} ( E ( t ) ) \frac{\partial }{\partial {μ}^{s}} s ( t , {μ}^{s} ) = - {α}^{s} s ( t , {μ}^{S} ) , \qquad Equation ( 5 )$$

$${v}^{s} ( E ( t ) ) s ( t , {μ}_{min}^{s} ) = r ( t , {μ}_{max}^{r} ) ,$$

$$s ( 0 , {μ}^{s} ) = {r}_{0} ( {μ}^{S} ) ,$$

where s(t, μ^{s}) is the reticulocytes population density at time t with maturity

$$18 = {μ}_{min}^{s} \leq {μ}^{s} \leq {μ}_{max}^{s} = 20.$$

Further on, ν^{s} is the maturation velocity depending on EPO (Assumption 12), as denotes the rate with which reticulocytes are phagocytosed (Assumption 13),

$${v}^{s} ( E ( t ) ) s ( t , {μ}_{min}^{s} ) = r ( t , {μ}_{max}^{r} )$$

describes the number of cells leaving the erythroblast cell stage and entering the reticulocyte cell stage, r₀(μ^{s}) is the population density at t=0.

A sigmoid function (e.g., Equation (6)) is used to describe the changes in maturation velocity ν^{s},

$${v}^{s} ( E ( t ) ) = \frac{{a}_{2} - {b}_{2}}{1 + {e}^{- {k}_{2} E ( t ) + {c}_{2}}} + {b}_{2} \qquad Equation ( 6 )$$

where E(t) is the EPO concentration at time t and a₂, b₂, c₂ and k₂ are positive constants with a₂>b₂. The maturation velocity increases with a rising concentration of EPO. Note, a slower maturation velocity causes the cells to reach the maximum cell age μ^{s}ₘₐₓ at a later point, whereas a faster maturation velocity shortens the transit time, i.e., the cells reach μ^{s}ₘₐₓ earlier.

### Erythrocytes

### Assumptions

14. Erythrocytes and blood reticulocytes are subsumed in one class.
15. Cells mature but do not proliferate.
16. There is a fixed random daily break-down of red blood cells (not to be confused with loss of erythrocytes due to senescence).
17. A drop in EPO concentration beneath a threshold precipitates neocytolysis.
18. Erythrocytes with age between 14-21 days are likely to be affected by neocytolysis.
19. Cells are phagocytosed when they reach the maximum age.
20. The maximum age of erythrocytes in a healthy person is 120 days.

The reticulocytes are released from the bone marrow into the blood stream and within 1-2 days they mature to erythrocytes. Circulating red blood cells have no nuclei and therefore they are not able to proliferate and they are not able to repair themselves. Thus, the life span of these cells is limited. In healthy adults the average life span is about 120 days, but it can significantly shorten in some pathologies. If not otherwise stated, 120 days may be used to be the maximal age of erythrocytes for the model.

Cells can be lost for different reasons:
due to internal or external bleeding,
because of random daily-breakdown,
because neocytolysis is triggered,
and last but not least, because of eryptosis of senescent cells.

Altogether, the following equation (e.g., Equation (7)) is obtained for red blood cells:

$$\frac{\partial }{\partial t} m ( t , {μ}^{m} ) + \frac{\partial }{\partial {μ}^{m}} m ( t , {μ}^{m} ) = - {α}^{m} ( E ( t ) , {μ}^{m} ) m ( t , {μ}^{m} ) , \qquad Equation ( 7 )$$

$$m ( t , 0 ) = {v}^{s} ( E ( t ) ) s ( t , {μ}_{max}^{s} ) ,$$

$$m ( 0 , {μ}^{m} ) = {m}_{0} ( {μ}^{m} ) ,$$

where m(t, μ^{m}) is the population density for the erythrocyte class at time t with maturity μ^{m}, t >0 and 0=μ^{m}ₘᵢₙ≥μ^{m}≥^{m}ₘₐₓ=120 (Assumption 20). Moreover,

$$m ( t , 0 ) = {v}^{s} ( E ( t ) ) s ( t , {μ}_{max}^{s} )$$

describes the number of reticulocytes entering the blood stream and m₀(μ^{m}) is the population density at t=0. In the population equation α^{m}(E(t), μ^{m}) denotes a random daily break-down and neocytolysis

$${α}^{m} ( E ( t ) , {μ}^{m} ) = \{ \begin{matrix}{γ}^{m} ( {μ}^{m} ) + min ( \frac{{c}_{E}}{{E ( t )}^{{k}_{E}}} , {b}_{E} ) & for E ( t ) < {τ}_{E} , {μ}_{min}^{m , n} \leq {μ}^{m} \leq {μ}_{max}^{m , n} \\ & \\ {γ}^{m} ( {μ}^{m} ) & otherwise\end{matrix} , \qquad Equation ( 8 )$$

where b_{E}, c_{E}, k_{E} are positive constants. τ_{E} is the threshold beneath which neocytolysis is triggered (Assumption 17), and

$$[ {μ}_{min}^{m , n} , {μ}_{max}^{m , n} ] = [ 14 , 21 ]$$

(Assumption 18) is the age interval during which cells are affected by neocytolysis, and t≥0. Since it is assumed that a cell is phagocytosed when it reaches its maximum age (Assumption 19), the mortality rate γ^{m}(μ^{m}) needs to be chosen such that

$${γ}^{m} ( {μ}^{m} ) = {α}_{r a n d}^{m} for {μ}_{min}^{m} \leq {μ}^{m} \leq {μ}_{max}^{m} - δ with δ > 0$$

sufficiently small and

$${\int }_{{μ}_{max - δ}^{m}}^{{μ}_{max}^{m}} {γ}^{m} ( {μ}^{m} ) = ∞ .$$

Here α^{r}_{rand} is a random daily break-down (Assumption 16).
A possible choice for γ^{m}(μ^{m}) is

$${γ}^{m} ( {μ}^{m} ) = \{ \begin{matrix}{α}_{r a n d}^{m} & for {μ}^{m} ∈ [ {μ}_{min}^{m} , {μ}_{ma x}^{m} - δ ] \\ \frac{3 {α}_{r a n d}^{m} {δ}^{2}}{{( {μ}^{m} )}^{2} - 2 ( {μ}_{ma x}^{m} + δ ) {μ}^{m} + ( {μ}_{ma x}^{m} + 2 δ ) {μ}_{ma x}^{m}} & for {μ}^{m} ∈ [ {μ}_{ma x}^{m} - δ , {μ}_{max}^{m} ] \\ ∞ & for {μ}^{m} \geq {μ}_{ma x}^{m}\end{matrix}$$

Additionally, it is possible to add a further term

$${α}_{b l e e d}^{m} ( t )$$

to the mortality rate in case of bleeding.

Erythropoietin Assumptions
21. Release of EPO is controlled by a negative feedback mechanism according to the oxygen content.
22. Oxygen carrying capacity is directly proportional to the number of erythrocytes.
23. The degradation rate of EPO is constant.
24. There is a slight delay in reaction of the EPO production rate to the number of RBC but this is negligible compared to the duration of development of erythrocytes.

The kidneys adjust the release of EPO according to the oxygen carrying capacity of the blood. If blood oxygen content is lower than normal, then EPO production increases and vice versa. Thus, the production of EPO is controlled by a negative feedback mechanism and allows for more red blood cells to be developed in case of an undersupply of the body with oxygen by opposing programmed cell death of erythroid progenitor cells. Additionally, if EPO decreases beneath a certain threshold, neocytolysis is triggered, the process wherein macrophages start to phagocytose young erythrocytes (neocytes).

A sigmoid function which depends on blood oxygen partial pressure is used to model the feedback involving the release of erythropoietin

$${E}_{i n}^{e n d} ( t )$$

from the kidneys into plasma. As a consequence of Assumption 22 the amount of

$${E}_{i n}^{e n d} ( t )$$

of EPO released by the kidney per unit time can be directly computed by use of the total population of erythrocyte M(t). Recall that this class consists of all circulating red blood cells (Assumption 14):

$${E}_{i n}^{end} ( t ) = \frac{{a}_{3} - {b}_{3}}{1 + {e}^{{k}_{3} \tilde{M} ( t ) - {c}_{3}}} + {b}_{3} ,$$

where \tilde{M}(t)=10⁻⁸M(t)/TBV is a scaled erythrocytes “concentration”. Note, that

$$M ( t ) = {\int }_{0}^{{μ}_{ma x}^{m}} m ( t , {μ}^{m} ) d {μ}^{m}$$

and TBV is the total blood volume. The constants a₃, b₃, c₃, k₃ are positive and satisfy a₃>b₃. The function

$${E}_{i n}^{e n d} ( t )$$

is monotonicany decreasing. Thus, the release of EPO increases if the number of circulating red blood cells decreases (Assumption 21). The dynamics of the endogenous EPO concentration E^{end}(t) in plasma are described by the following ordinary differential equation:

$$\frac{d}{d t} {E}^{end} ( t ) = \frac{1}{T B V} {E}_{i n}^{end} ( t ) - {c}_{deg}^{end} {E}^{end} ( t ) ,$$

where E^{end}(t) is the endogenous EPO concentration in plasma, E^{end}ᵢₙ is the amount of EPO released by the kidneys and

$${c}_{d e g}^{e n d}$$

describes the constant degradation rate of endogenous EPO (Assumption 23).

The degradation rate for exogenous

$$EPO \qquad {c}_{d e g}^{e x}$$

differs from the one for endogenous EPO and varies according to the kind of ESA administered. Therefore, an additional ODE is needed to describe the change in the plasma concentration of an ESA

$$\frac{d}{d t} {E}^{e x} ( t ) = \frac{1}{T B V} {E}_{i n}^{e x} ( t ) - {c}_{deg}^{e x} {E}^{e x} ( t ) ,$$

where

$${E}_{i n}^{e x} ( t )$$

is the rate at which the artificial hormone is administered and

$${c}_{d e g}^{e x}$$

is the rate with which the exogenous hormone is degraded. In intravenous administration, the total amount of the agent is injected into a vein, within a very short time interval. In this case

$${E}_{in}^{ex} ( t )$$

can be approximated by

$${E}_{0}^{ex} ( t ) {δ}_{{t}_{0}} ( t ) ,$$

where

$${E}_{0}^{ex}$$

is the amount of artificial hormone administered and δₜ_{₀}(t) is the Dirac delta impulse located at t₀, the time when the administration takes place. The overall concentration of EPO in blood consists of the naturally produced erythropoietin in the body and the administered ESA

$$E ( t ) = {E}^{ex} ( t ) + {E}^{in} ( t ) .$$

### Assumptions Revisited

25. The number of cells which commit to the erythroid lineage, is EPO dependent.
26. Cells normally stay in this stage for 8 days (3 days BFU-E and 5 days CFU-E).
27. Under high levels of EPO, stress reticulocytes are released.

It is recommended to change Assumption 1 and 2 to Assumption 25 and 26. The number of stems cells which become BFU-E cells per unit time can then be described by a sigmoidal function

$${S}_{0} ( t ) = \frac{{a}_{4} - {b}_{4}}{1 + {e}^{- {k}_{4} E ( t ) + {c}_{4}}} + {b}_{4} ,$$

where E(t) is the EPO concentration and a₄, b₄, c₄ and k₄ are constants. The definition of a₄=1.2b₄ may be used because there is evidence that the maximum stem cell increase can be by about 20%. Assumption 26 implies that the cell age ranges for progenitor cells have to be changed to

$${μ}_{max}^{p} = {μ}_{min}^{q} = 3 and {μ}_{max}^{q} = 8 .$$

Further Assumption 27 should be added to the list of assumptions for precursor cells above. This assumption can be understood as follows: when the EPO concentration increases above a certain threshold τₛ the whole reticulocyte population class is released to the bloodstream and the class is skipped, i.e., cells that mature to marrow reticulocytes are immediately released from the bone marrow, as long as EPO levels remain above the threshold.

### COMPUTATION OF HEMATOCRIT AND HEMOGLOBIN CONCENTRATIONS

In order to compute the hematocrit (HCT) and the hemoglobin concentration (Hb) for a subject from the model output (which is the number of red blood cells circulating in blood), estimates of the total blood volume of the subject are needed. The Nadler and Allen formula can be used to estimate the total blood volume (TBV) of a healthy subject according to her/his weight and height:

$$TBV [ ml ] = 183.3 + 356.1 \times {( height [ m ] )}^{3} + 33.08 \times weight [ kg ] , : TBV [ ml ] = 604.1 + 366.9 \times {( height [ m ] )}^{3} + 32.19 \times weight [ kg ] .$$

The TBV for a dialysis patient can be measured by radio-labeling red blood cells with chromium-51 as described above. Using these estimates for the total blood volume, the hematocrit (HCT) and the hemoglobin concentration (Hb) can be computed for a patient from the number of red blood cells circulating in blood via the following formulae:

$$HCT [ \% ] = \frac{( M ( t ) \times MCV [ fl ] )}{TBV [ ml ]} ,$$

where

$$M ( t ) = {\int }_{0}^{{μ}_{max}^{m}} m ( t , {μ}^{m} ) d {μ}^{m}$$

is the total number of erythrocytes circulating in blood and MCV is the mean corpuscular volume of a RBC which is obtained from measurement, and

$$Hb [ \frac{g}{1} ] = 1000 \times \frac{M ( t ) \times MCH [ pg ]}{TBV [ ml ]} ,$$

where MCH is the mean cellular hemoglobin, which is also obtained via measurements.

### EXPERIMENTAL RESULTS

Experiments using hemoglobin time series and models according to some embodiments were conducted to determine the effects of individualized anemia therapy on hemoglobin stability, for example, via a randomized controlled pilot trial of hemodialysis patients. The experiments involved a multi-center, randomized, controlled trial comparing the anemia therapy assistance system and associated software against a standard population-based anemia treatment protocol. The results of the experiments demonstrated that, inter alia, personalized dosing of erythropoiesis stimulating agents (ESA) using models and processes according to various embodiments improves hemoglobin target attainment.

In general, the experimental methods included a patient cohort of ninety-six patients undergoing hemodialysis and receiving methoxy polyethylene glycol-epoetin beta randomized 1:1 to the intervention group (personalized ESA dose recommendations computed by the software configured according to some embodiments) or the standard of care group for twenty-six weeks. The therapy assistance software configured according to some embodiments combined a physiology-based mathematical model, a model predictive controller designed to stabilize hemoglobin levels within a tight target range (10 and 11 g/dl), and hemoglobin time series processes according to various embodiments. The primary outcome measure was the percentage of hemoglobin measurements within the target. Secondary outcome measures included measures of hemoglobin variability and ESA utilization.

The intervention group showed an improved median percentage of hemoglobin measurements within target at 47% (IQR 39 to 58), with a 10 percentage points median difference between the two groups (95% CI: 3 to 16; P=0.008). The odds ratio of being within the hemoglobin target in the standard of care group compared to the group receiving the personalized ESA recommendations (using models and hemoglobin time series data according to various embodiments) was 0.7 (95% CI: 0.5 to 0.9). The variability of hemoglobin levels decreased in the intervention group, with the percentage of patients experiencing fluctuating hemoglobin levels being 45% vs 82% in the standard of care group. ESA usage was reduced by about 25% in the intervention group.

The results demonstrated an improved hemoglobin target attainment and variability by employing personalized ESA recommendations using the physiology-based anemia therapy assistance software including models and hemoglobin time series processes according to some embodiments.

Participants in the standard of care arm continued to be treated per standard anemia therapy protocol used in clinics. This rule-based protocol was the same in all participating clinics. It consists of a set of prewritten guidelines reviewed by the LDO's medical advisory board. The treatment protocol takes up to the latest three hemoglobin measurements into account. Adjustments to the current ESA dose are suggested based on the most recent hemoglobin level and the trend of the hemoglobin curve.

The intervention group used models and hemoglobin time series information according to various embodiments. For participants in the intervention arm, the therapy assistance software (i.e., software 102) computed individualized ESA dose recommendations fortnightly. In general, the software combines a generic physiology-based model, an algorithm to individualize the model based on patient's routine clinical data, and a model predictive controller. It is designed to guide and stabilize a patient's hemoglobin level within a predefined target range.

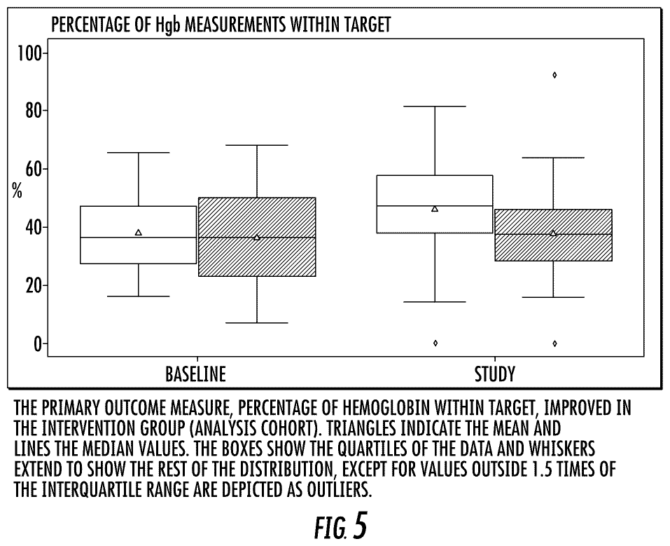

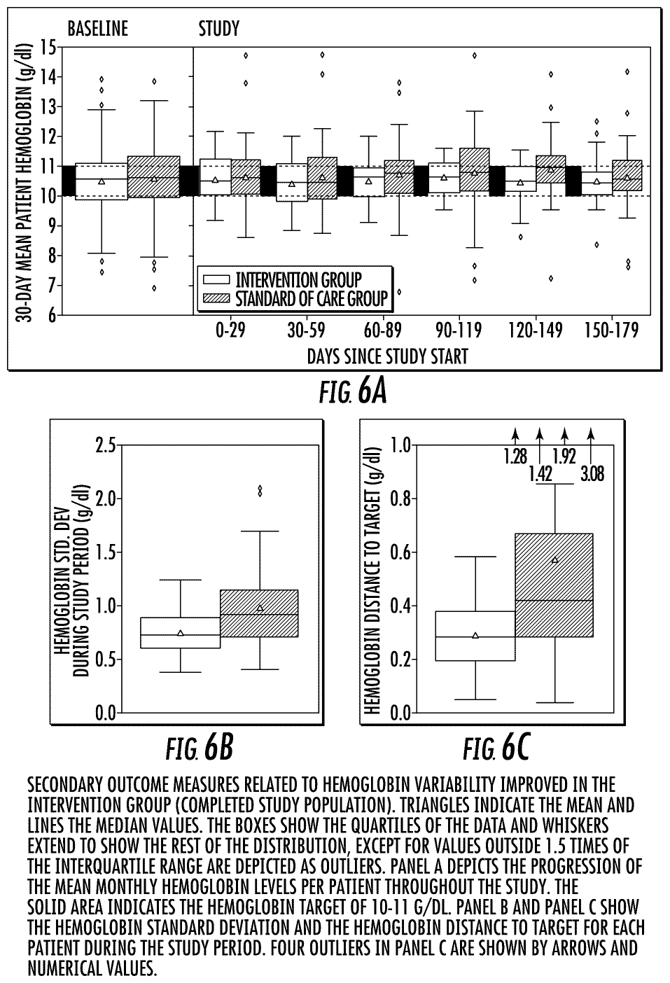

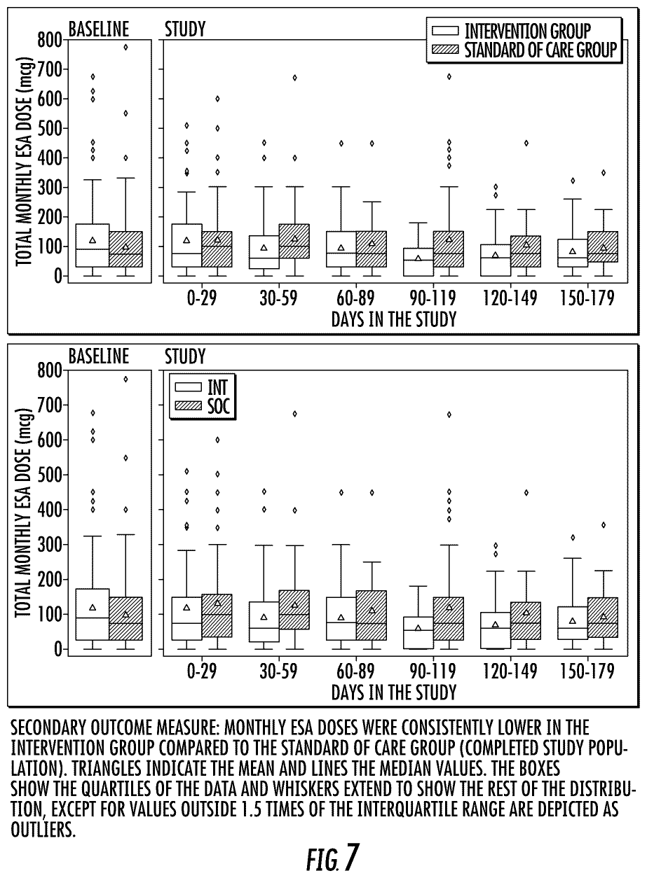

Patient cohort information and experimental results are provided in FIG. 5, FIGS. 6A-6C, and FIG. 7 and the following Tables 1-3:

TABLE 1
Baseline characteristics of all randomized study participants. Data are given as
count (percentage) or median (interquartile range) as appropriate. ESA, erythropoiesis
stimulating agent;; spKt/V, single pool Kt/V; PTH, parathyroid hormone.

| | | | Standard of | | | |
|---|---|---|---|---|---|---|
| | Intervention group, | | care group, | | Total, | |
| Characteristic | n = 49 | | n = 47 | | n = 96 | |
| Age, years | 62 | (55, 71) | 60 | (50, 72) | 61 | (51) |
| Men | 31 | (63) | 27 | (57) | 58 | (60) |
| Dialysis vintage, | 3 | (2, 7) | 3 | (1, 6) | 3 | (1, 7) |
| years | | | | | | |
| Dialysis access | | | | | | |
| type, n (%): | | | | | | |
| Arteriovenous | 31 | (63) | 32 | (68) | 63 | (66) |
| fistula | | | | | | |
| Arteriovenous | 8 | (16) | 8 | (17) | 16 | (17) |
| graft | | | | | | |
| Central venous | 10 | (20) | 8 | (17) | 18 | (19) |
| catheter | | | | | | |
| Body mass index, | 26.9 | (21.6, 30.5) | 26.3 | (22.5, 30.8) | 26.5 | (22.1, 30.7) |
| kg/m2 | | | | | | |
| Post-dialysis | 74 | (59, 83) | 70 | (60, 82) | 71 | (59, 83) |
| body weight, kg | | | | | | |
| Interdialytic | 2.2 | (1.7, 2.7) | 2.2 | (1.9, 2.8) | 2.2 | (1.7, 2.8) |
| weight gain, kg | | | | | | |
| Hemoglobin at | 11.0 | (10, 11.8) | 10.7 | (9.5, 12.0) | 10.9 | (9.8, 11.8) |
| end of baseline, | | | | | | |
| g/dl | | | | | | |
| Mean | 10.5 | (10.1, 11) | 10.7 | (10.3, 10.9) | 10.6 | (10.2, 10.9) |
| hemoglobin | | | | | | |
| during baseline, | | | | | | |
| g/dl | | | | | | |
| Hemoglobin | 0.9 | (0.8, 1) | 0.9 | (0.8, 1.4) | 0.9 | (0.8, 1.3) |
| standard | | | | | | |
| deviation, g/dl | | | | | | |
| Hemoglobin | 36 | (27, 46) | 38 | (24, 49) | 37 | (26, 47) |
| values in target, | | | | | | |
| % | | | | | | |
| Hemoglobin | 0.4 | (0.3, 0.6) | 0.4 | (0.3, 0.7) | 0.5 | (0.3, 0.7) |
| distance to | | | | | | |
| target, g/dl | | | | | | |
| Mean monthly | 93 | (55, 176) | 86 | (60, 130) | 90 | (58, 143) |
| ESA dose, | | | | | | |
| mcg/30 days | | | | | | |
| Rate of ESA dose | 1.3 | (1.0, 1.7) | 1.3 | (1.0, 1.5) | 1.3 | (1.0, 1.5) |
| administrations, | | | | | | |
| 1/30 days | | | | | | |
| Iron dose, mg/30 | 208 | (75, 292) | 225 | (129, 292) | 213 | (108, 292) |
| days | | | | | | |
| Ferritin, ng/ml | 805 | (484, 1067) | 893 | (499, 1265) | 839 | (490, 1168) |
| Transferrin | 32 | (25, 37) | 32 | (27, 41) | 32 | (26, 38) |
| saturation, % | | | | | | |
| Serum albumin, | 4.0 | (3.8, 4.1) | 3.9 | (3.7, 4.1) | 3.9 | (3.7, 4.1) |
| g/dl | | | | | | |
| Neutrophiles-to- | 3.2 | (2.4, 4.8) | 3.3 | (2.7, 4.1) | 3.2 | (2.4, 4.8) |
| lymphocytes ratio | | | | | | |
| spKt/V | 1.7 | (1.5, 1.9) | 1.7 | (1.6, 1.8) | 1.7 | (1.5, 1.9) |
| Intact PTH, | 669 | (403, 928) | 507 | (339, 726) | 546 | (385, 796) |
| pg/ml | | | | | | |
| Hospitalizations | 0 | (0, 1) | 0 | (0, 1) | 0 | (0, 1) |
| per 180 days | | | | | | |
| Hospitalization | 5 | (4, 7) | 3 | (2, 5) | 4 | (2, 6) |
| duration per | | | | | | |
| episode, days | | | | | | |
| Diabetes | | | | | | |
| mellitus, n (%) | | | | | | |
| No | 27 | (55) | 15 | (32) | 42 | (44) |
| Yes | 22 | (45) | 32 | (68) | 54 | (56) |
| Congestive heart | | | | | | |
| failure, n (%) | | | | | | |
| No | 41 | (84) | 41 | (87) | 82 | (85) |
| Yes | 8 | (16) | 6 | (13) | 14 | (15) |

TABLE 2
Primary and secondary outcome measures (analytical cohort). Data are
given as median (interquartile range). Bootstrapping was used to calculate
the median difference and the 95% confidence interval of the difference.
Group comparisons were performed by Wilcoxon rank-sum and Chi-squared
test as appropriate. ESA, erythropoiesis stimulating agent.

| | | Standard of | |
|---|---|---|---|
| | Intervention group, | care group, | Median Difference |
| Characteristic | n = 46 | n = 45 | (95% CI); P-value |

Primary outcome
measure

| Hemoglobin values | 47 | (39, 58) | 38 | (29, 46) | 10 (3, |
|---|---|---|---|---|---|
| in target, % | | | | | 16); 0.008 |
| Secondary outcome | | | | | |
| measures | | | | | |
| Patients with | 45 | | 82 | | −37 (−54, |
| fluctuating | | | | | −17); 0.0009 |
| hemoglobin | | | | | |
| levels, % | | | | | |
| (Intervention | | | | | |
| group: n = 44, | | | | | |
| standard of care | | | | | |
| group: n = 44) | | | | | |
| Mean hemoglobin, | 10.4 | (10.2, 10.7) | 10.7 | (10.4, 11.1) | −0.3 (−0.4, |
| g/dl | | | | | −0.008); 0.03 |
| Hemoglobin | 0.7 | (0.6, 0.9) | 0.9 | (0.7, 1.1) | −0.2 (−0.3, |
| standard | | | | | −0.04); 0.0005 |
| deviation, g/dl | | | | | |
| Hemoglobin | 0.3 | (0.2, 0.4) | 0.4 | (0.3, 0.7) | −0.2 (−0.3, |
| distance to | | | | | −0.05); 0.0006 |
| target, g/dl) | | | | | |
| Mean ESA dose, | 69 | (47, 114) | 91 | (60, 148) | −23 (−67, |
| mcg/30 days | | | | | 5); 0.055 |

| | | | |
|---|---|---|---|
| Mean ESA dose, | 1.1 | 1.5 | −0.4 (−0.8, |
| mcg/30 days/kg | | | 0.1); 0.02 |

TABLE 3
Other Outcomes (analytical cohort). Data are presented as median (inter quartile
range). Bootstrapping was used to calculate the median difference and the
95% confidence interval of the difference. Group comparisons were performed
by Wilcoxon rank-sum test.; ESA, erythropoiesis stimulating agent.

| | | | Standard of | | |
|---|---|---|---|---|---|
| | Intervention group, | | care group, | | Median Difference |
| Characteristic | n = 46 | | n = 45 | | (95% CI); P-value |
| Rate of ESA | 1.4 | (0.8, 1.6) | 1.3 | (1, 1.6) | 0.03 (−0.3, |
| administration, | | | | | 0.3); 0.9 |
| per 30 days | | | | | |
| Iron dose, | 163 | (58, 229) | 142 | (75, 225) | 25 (−50, |
| mg/30 days | | | | | 83); 0.9 |
| Ferritin | 849 | (567, 1053) | 765 | (608, 1249) | 70 (−277, |
| concentration, | | | | | 212); 0.5 |
| ng/ml | | | | | |
| Transferrin | 29 | (25, 39) | 33 | (27, 45) | −4 (−11, |
| saturation, % | | | | | 2); 0.1 |
| (Intervention | | | | | |
| group: n = 41; | | | | | |
| standard of care | | | | | |
| group: n = 43) | | | | | |

### IMPLEMENTATION IN A COMPUTER NETWORK

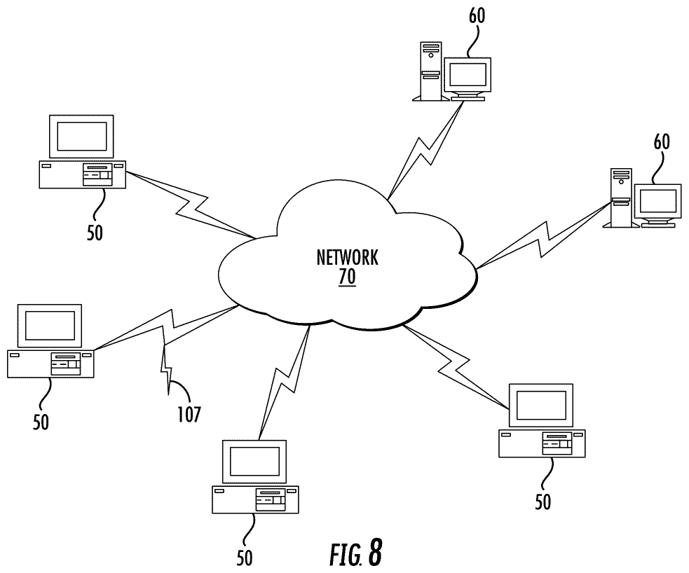

FIG. 8 illustrates a computer network or similar digital processing environment in which the described embodiments may be implemented.

Computer(s)/devices 50 and server computer(s) 60 provide processing, storage, and input/output devices executing application programs and the like. Computer(s)/devices 50 can also be linked through communications network 70 to other computing devices, including other devices/processes 50, digital processor dialysis machines 50A, and server computer(s) 60. Communications network 70 can be part of a remote access network, a global network (e.g., the Internet), a worldwide collection of computers, Local area or Wide area networks, and gateways that currently use respective protocols (TCP/IP, Bluetooth, etc.) to communicate with one another. Other electronic device/computer network architectures are suitable.

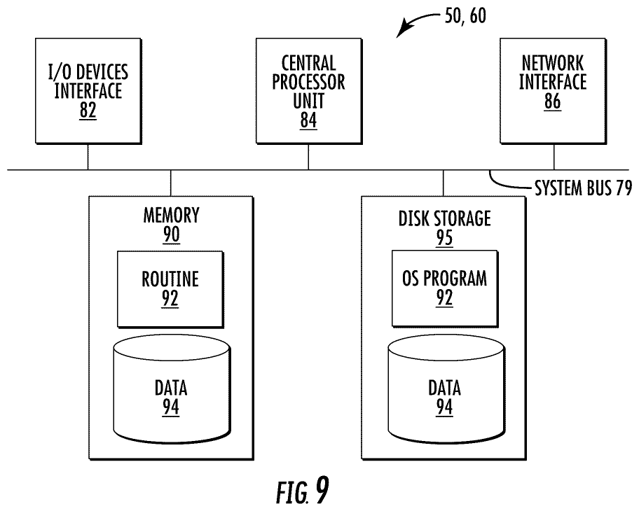

FIG. 9 is a diagram of the internal structure of a computer (e.g., processor/device 50, digital processor dialysis machines 50A, or server computers 60) in the computer system of FIG. 8. Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., keyboard, mouse, displays, printers, speakers, etc.) to the computer 50, 60. Network interface 86 allows the computer to connect to various other devices attached to a network (e.g., network 70 of FIG. 8). Memory 90 provides volatile storage for computer software instructions 92 and data 94 used to implement some embodiments (e.g., equations. shown in FIG. 3 or any other erythropoiesis modeling engine detailed above). Disk storage 95 provides non-volatile storage for computer software instructions 92 and data 94 used to implement some embodiments. Central processor unit 84 is also attached to system bus 79 and provides for the execution of computer instructions.

In one embodiment, the processor routines 92 and data 94 are a computer program product (generally referenced 92), including a computer readable medium (e.g., a removable storage medium such as one or more DVD-ROM's, CD-ROM's, diskettes, tapes, etc.) that provides at least a portion of the software instructions for the system. Computer program product 92 can be installed by any suitable software installation procedure, as is well known in the art. In another embodiment, at least a portion of the software instructions may also be downloaded over a cable, communication and/or wireless connection. In other embodiments, the programs are a computer program propagated signal product 107 embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present embodiments routines/program 92.

In alternate embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium and the like.

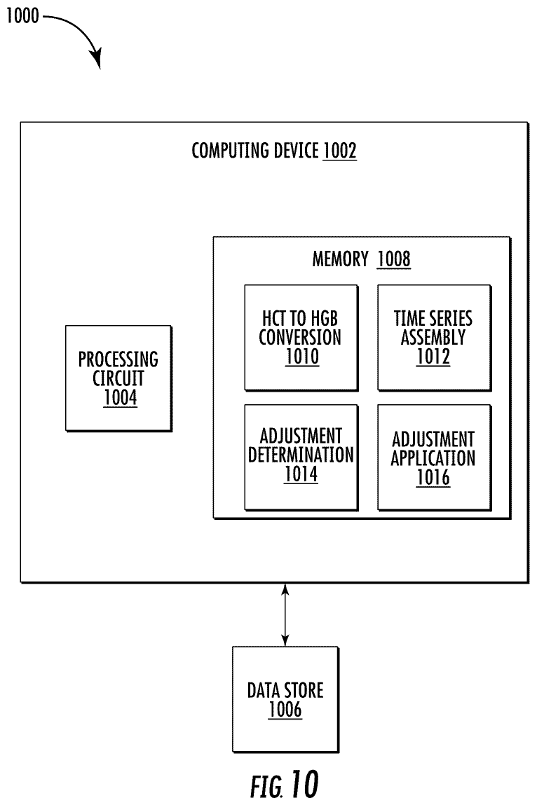

FIG. 10 illustrates a block diagram of a computing environment 1000 that includes computing device 1002 and data store 1006. In some embodiments, the computing device 1002 can be any suitable computing device such as a computer, a laptop, a desktop computer, a mobile work station, a server, a cloud server, a mobile device, or any other suitable computing device capable of performing operations described herein. In some embodiments, the data store 1006 can be a server, a cloud server, a hard drive, a storage device, a storage network device, a database, a flash drive, or any other data store now known or later discovered.

In some embodiments, the computing device 1002 comprises a processing circuit 1004 and memory 1008. The processing circuit 1004 may include any suitable processing circuit such as a processor, a central processing unit (CPU), microprocessor, graphics processing unit (GPU), controller, microcontroller, application specific integrated circuit (ASIC), field programmable gate array (FPGA), or any other suitable processing circuit now known or later discovered. The memory 1008 can include any suitable memory 1008 such as random-access memory (RAM), read only memory (ROM), a hard drive, a solid state drive, a flash drive, flash memory, or any other suitable memory now known or later discovered.

In some embodiments, the computing device 1002 is configured to generate an aligned hemoglobin time series for a patient using multiple measurement sources. For example, in some embodiments, the memory 1008 can include executable instructions stored thereon, which when executed by the processing circuit 1004, causes the processing circuit to perform various operations. In some embodiments, the processing circuit 1004 is configured to access hemoglobin measurements (Hgb) for the patient from the data store 1006. Moreover, in some embodiments, the processing circuit 1004 is configured to access hematocrit measurements (HCT) for the patient measured during treatments on the patient. From the accessed HCT measurements, the processing circuit 1004 is configured to identify the hematocrit measurements that correspond to the start (pre HCT) and end (post HCT) of the treatments.

In some embodiments, the memory 1008 includes an application for HCT to HGB conversion 1010. The processing circuit 1004 is configured to execute the HCT to HGB conversion application 1010 and convert the pre HCT and post HCT to hemoglobin values pre Hgb and post Hgb. The memory 1008 further includes a time series assembly application 1012, which the processing circuit 1004 executes to assemble a hemoglobin time series of the Hgb, pre Hgb, and post Hgb for the patient. The memory 1008 further includes an adjustment determination application 1014, which the processing circuit 1004 executes to determine an adjustment to values of the pre Hgb and values of the post Hgb to improve alignment with values of the Hgb.

In some embodiments, the memory 1008 further includes an adjustment application 1016, which the processing circuit executes to apply the adjustment to the values of the pre Hgb and the values of the post Hgb, yielding aligned pre Hgb values and aligned post Hgb values. Next, the processing circuit 1004 is configured to populate the aligned hemoglobin time series for a desired time window using Hgb during treatment sessions when Hgb data is available within the desired time window. In some embodiments, the processing circuit 1004 is configured to use aligned pre Hgb values and aligned post Hgb values to populate the aligned hemoglobin time series during treatment sessions when Hgb data is unavailable.

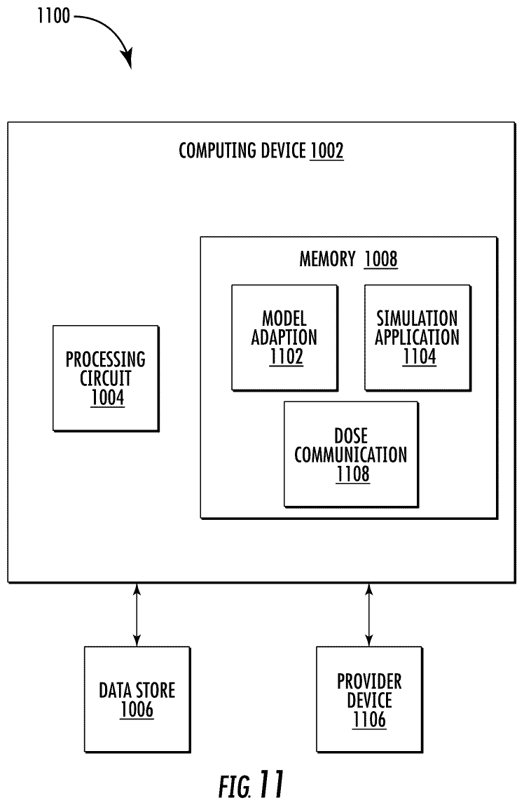

The aligned hemoglobin time series can be fed into a predictive model such as the models described in FIG. 11 to recommend an ESA dose for treating anemia in patients. The aligned hemoglobin time series provides a smoothed time series of hemoglobin data that the predictive models and simulations in FIG. 11 can efficiently and quickly process to provide the recommended ESA dose. This process provides one of the improvements discussed above over existing systems that make the predictive models and simulations more efficient because they do not have to process the noisy hemoglobin and hematocrit measurements of patients.

In some embodiments, the processing circuit 1004 is configured to selectively filter the hemoglobin measurements (Hgb), wherein the filtering excludes Hgb values that are outside a first threshold from a first moving average. In some other embodiments, the processing circuit is configured to selectively filter the pre HCT and post HCT, wherein the filtering excludes hematocrit measurements that are outside a second threshold from a second moving average for the hematocrit measurements.

In some embodiments, determining the adjustment includes the processing circuit 1004 being configured to determine an offset correction by computing a mean offset between the pre Hgb values and the Hgb values, and subtracting the mean offset from the pre Hgb values and post Hgb values to yield offset pre Hgb values and offset post Hgb values. In some embodiments, determining the adjustment includes the processing circuit 1004 being configured to determine an interpolation factor between offset pre Hgb values and offset post Hgb values such that the average deviation between a resulting interpolated hemoglobin and the Hgb is minimized.

In some embodiments, applying the adjustment to the pre Hgb values and post Hgb values includes the processing circuit 1004 being configured to subtract the mean offset from the pre Hgb values and post Hgb values and applying the interpolation factor yielding the aligned pre Hgb values and aligned post Hgb values. In some embodiments, the hemoglobin measurements (Hgb) are from laboratory hemoglobin measurements performed on blood samples of the patient. In some other embodiments, the hematocrit measurements (HCT) are measured by non-invasive photo-optical sensors during extracorporeal treatments on the patient.

In some embodiments, the hematocrit measurements that correspond to the end of a treatment that was ended unexpectedly are disregarded. In some embodiments, the patient is a dialysis patient and the hematocrit measurements are collected as part of the patient's regular dialysis treatments. In some embodiments, the aligned hemoglobin time series spans a period of at least 90 days and includes at least 15 values. In some embodiments, the processing circuit 1004 is configured to feed the aligned hemoglobin time series into a predictive model to determine an erythropoiesis-stimulating agent (ESA) dose for the patient to assist with treating anemia in the patient.

FIG. 11 is a block diagram of another computing environment 1100 that includes the computing device 1002 of FIG. 10, along with the data store 1006. The computing environment 1100 is configured to run simulations or predictive models to assist in treating anemia in patients. The computing device 1002 of FIG. 11 further includes the processing circuit 1004 and memory 1008 of the computing device 1002 of FIG. 10. However, in this embodiment, the memory 1008 includes a model adaption application 1102, simulation application 1104, and a dose communication application 1108 with instructions for executing those applications as well. Furthermore, the computing device 1002 is in communication with a provider device 1106.

In some embodiments, the provider device 1106 can include a computer, a desktop, a computing device, a mobile device, a tablet computer, a mobile workstation, a laptop, a server, a cloud server, or any other suitable computing device that can receive communications described herein.

In some embodiments, the processing circuit 1004 is configured to communicate with the data store 1006 to access patient parameters associated with the patient, wherein the patient parameters include an aligned hemoglobin time series for the patient generated from multiple measurement sources. Furthermore, the processing circuit 1004 is configured to access a physiology-based model from the data store 1006.

In some embodiments, the processing circuit 1004 is configured to execute the model adaption application 1102 to thereby adapt the physiology-based model into a patient specific model that predicts future hemoglobin levels for the patient based on one or more erythropoiesis-stimulating agent (ESA) dosing regimens. In some embodiments, adapting the physiology-based model to the patient specific model includes the processing circuit 1004 being configured to utilize the patient parameters, and generate estimates of patient-specific physiological characteristics.

In some embodiments, the processing circuit 1004 is configured to run simulations with the patient specific model and determine a recommended ESA dose that the model predicts will cause either or both of the patient's hematocrit or hemoglobin concentration to reach a desired range within a specified time frame. In some embodiments, the processing circuit 1004 is configured to execute the dose communication application 1108 causing the processing circuit 1004 to be configured to provide or send the recommended ESA dose to the patient's healthcare provider. For example, the processing circuit 1004 can send the recommended ESA dose to the provider device 1106 for the healthcare provider to administer the dose.

In some embodiments, the aligned hemoglobin time series is generated by the processing circuit 1004 using the process described above in FIG. 10. In some embodiments, the processing circuit 1004 is further configured to generate a graph of the predicted hemoglobin trend for the patient associated with the recommended ESA dose, and provide or send the graph to the patient's healthcare provider (e.g., to the provider device 1106) with the recommended ESA dose.

In some embodiments, the patient parameters further comprise sex, height, post-hemodialysis weight, and historical ESA doses for the patient. In some embodiments, the patient specific model includes a plurality of patient specific models each with a unique set of patient-specific physiological characteristics. Moreover, in some embodiments, simulations are run by the processing circuit 1004 or any other suitable computing device on all of the models to determine the recommended ESA dose. In some embodiments, the aligned hemoglobin time series spans a period of at least 90 days and includes at least 15 values.

In some embodiments, adapting the physiology-based model into a patient specific model includes the processing circuit 1004 being configured to compare hemoglobin values from the aligned hemoglobin time series to hemoglobin predictions output by the patient-specific model, and a threshold for the patient specific model being valid is a mean percentage error of less than about 6%. In some embodiments, the patient-specific physiological characteristics include a red blood cell life span, an endogenous erythropoietin production, an ESA half-life, an ESA dependent apoptosis rate of erythrocyte progenitor cells, and an ESA dependent maturation function of erythrocyte precursor cells.

In some embodiments, an updated recommended ESA dose is provided at frequency of about every two weeks. In some embodiments, the desired range for the patient's hemoglobin is about 10-11 g/dl. In some embodiments, the specified time frame is at least about 8 weeks. In some embodiments, the patient's aligned hemoglobin time series is classified as “fluctuating” if either: the difference between the maximum hemoglobin value and the minimum hemoglobin value is larger than 1.75 g/dL, or a portion of time that a weekly hemoglobin rate of change exceeds 0.1 g/DL/week is larger than 60% for the aligned hemoglobin time series.

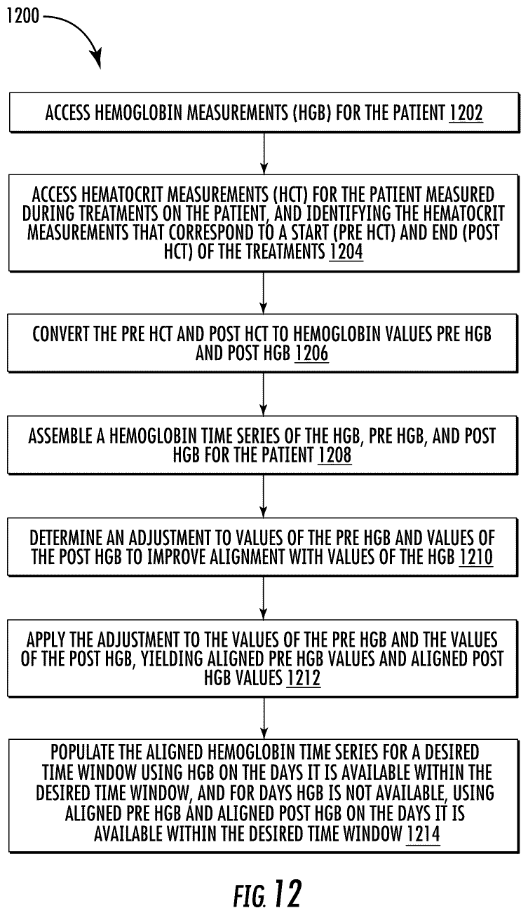

FIG. 12 is a flow chart illustrating example operations performed in a method 1200 includes for generating an aligned hemoglobin time series for a patient from multiple measurement sources. As shown at block 1202, the method 1200 includes accessing hemoglobin measurements (Hgb) for the patient. As shown at block 1204, the method 1200 includes accessing hematocrit measurements (HCT) for the patient measured during treatments on the patient, and identifying the hematocrit measurements that correspond to a start (pre HCT) and end (post HCT) of the treatments. As shown at block 1206, the method 1200 includes converting the pre HCT and post HCT to hemoglobin values pre Hgb and post Hgb. As shown at block 1208, the method 1200 includes assembling a hemoglobin time series of the Hgb, pre Hgb, and post Hgb for the patient.

As shown at block 1210, the method 1200 includes determining an adjustment to values of the pre Hgb and values of the post Hgb to improve alignment with values of the Hgb. As shown at block 1212, the method 1200 includes applying the adjustment to the values of the pre Hgb and the values of the post Hgb, yielding aligned pre Hgb values and aligned post Hgb values. As shown at block 1214, the method 1200 includes populating the aligned hemoglobin time series for a desired time window using Hgb on the days it is available within the desired time window, and for days Hgb is not available, using aligned pre Hgb and aligned post Hgb on the days it is available within the desired time window.

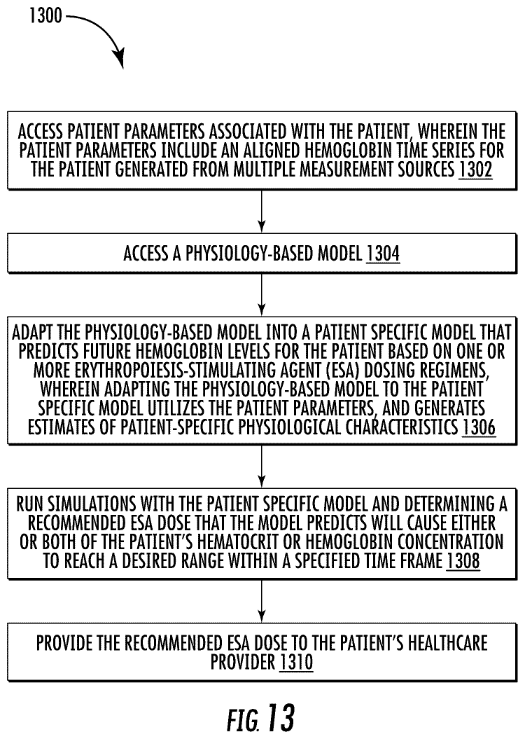

FIG. 13 is a flow chart illustrating example operations performed in another method 1300. As shown at block 1302, the method 1300 includes accessing patient parameters associated with the patient, wherein the patient parameters include an aligned hemoglobin time series for the patient generated from multiple measurement sources. As shown at block 1304, the method 1300 includes accessing a physiology-based model. As shown at block 1306, the method 1300 includes adapting the physiology-based model into a patient specific model that predicts future hemoglobin levels for the patient based on one or more erythropoiesis-stimulating agent (ESA) dosing regimens, wherein adapting the physiology-based model to the patient specific model utilizes the patient parameters, and generates estimates of patient-specific physiological characteristics.

As shown at block 1308, the method 1300 includes running simulations with the patient specific model and determining a recommended ESA dose that the model predicts will cause either or both of the patient's hematocrit or hemoglobin concentration to reach a desired range within a specified time frame. As shown at block 1310, the method 1300 includes providing the recommended ESA dose to the patient's healthcare provider.

Some embodiments of the disclosed system may be implemented, for example, using a storage medium, a computer-readable medium or an article of manufacture which may store an instruction or a set of instructions that, when executed by a machine (e.g., processor, processing circuit, or microcontroller), may cause the machine to perform a method and/or operations in accordance with embodiments of the disclosure. In addition, a server or database server may include machine readable media configured to store machine executable program instructions. Such a machine may include, for example, any suitable processing platform, computing platform, computing device, processing device, computing system, processing system, computer, processor, or the like, and may be implemented using any suitable combination of hardware, software, firmware, or a combination thereof and utilized in systems, subsystems, components, or sub-components thereof.

The various elements of the devices as previously described with reference to the figures above may include various hardware elements, software elements, or a combination of both. Examples of hardware elements may include devices, logic devices, components, processors, microprocessors, circuits, processors, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements may include software components, programs, applications, computer programs, application programs, system programs, software development programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. However, determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given implementation.

One or more aspects of at least one embodiment may be implemented by representative instructions stored on a non-transitory machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Some embodiments may be implemented, for example, using a machine-readable medium or article which may store an instruction or a set of instructions that, if executed by a machine, may cause the machine to perform a method and/or operations in accordance with the embodiments. Such a machine may include, for example, any suitable processing platform, computing platform, computing device, processing device, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

The foregoing description of example embodiments has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner, and may generally include any set of one or more limitations as variously disclosed or otherwise demonstrated herein.

What is claimed is:

1. A method of assisting with the management of anemia in a patient, comprising:
accessing patient parameters associated with the patient, wherein the patient parameters include an aligned hemoglobin time series for the patient generated from multiple measurement sources;
accessing a physiology-based model;
adapting the physiology-based model into a patient specific model that predicts future hemoglobin levels for the patient based on one or more erythropoiesis-stimulating agent (ESA) dosing regimens, wherein adapting the physiology-based model to the patient specific model utilizes the patient parameters, and generates estimates of patient-specific physiological characteristics;
running simulations with the patient specific model and determining a recommended ESA dose that the model predicts will cause either or both of the patient's hematocrit or hemoglobin concentration to reach a desired range within a specified time frame; and
providing the recommended ESA dose to the patient's healthcare provider.

2. The method of claim 1, wherein the aligned hemoglobin time series is generated by:
accessing hemoglobin measurements (Hgb) for the patient;
accessing hematocrit measurements (HCT) for the patient measured during treatments on the patient, and identifying the hematocrit measurements that correspond to a start (pre HCT) and end (post HCT) of the treatments;
converting the pre HCT and post HCT to hemoglobin values pre Hgb and post Hgb;
assembling a hemoglobin time series of the Hgb, pre Hgb, and post Hgb for the patient;
determining an adjustment to the pre Hgb and post Hgb to improve alignment with Hgb;
applying the adjustment to pre Hgb and post Hgb, yielding aligned pre Hgb and aligned post Hgb; and
populating the aligned hemoglobin time series for a desired time window using Hgb on days it is available within the desired time window, and for days Hgb is not available, using aligned pre Hgb and aligned post Hgb on the days it is available within the desired time window.

3. The method of claim 1, further comprising generating a graph of the predicted hemoglobin trend for the patient associated with the recommended ESA dose, and providing the graph to the patient's healthcare provider with the recommended ESA dose.

4. The method of claim 1, wherein the patient parameters further comprise sex, height, post-hemodialysis weight and historical ESA doses for the patient.

5. The method of claim 1, wherein the patient specific model includes a plurality of patient specific models each with a unique set of patient-specific physiological characteristics; and
wherein simulations are run on all of the models to determine the recommended ESA dose.

6. The method of claim 1, wherein the aligned hemoglobin time series spans a period of at least 90 days and includes at least 15 values.

7. The method of claim 1, wherein adapting the physiology-based model into a patient specific model includes comparing hemoglobin values from the aligned hemoglobin time series to hemoglobin predictions output by the patient-specific model, and a threshold for the patient specific model being valid is a mean percentage error of less than about 6%.

8. The method of claim 1, wherein the patient-specific physiological characteristics include a red blood cell life span, an endogenous erythropoietin production, an ESA half-life, an ESA dependent apoptosis rate of erythrocyte progenitor cells, and an ESA dependent maturation function of erythrocyte precursor cells.

9. The method of claim 1, wherein:
an updated recommended ESA dose is provided at frequency of about every two weeks;
the desired range for the patient's hemoglobin is about 10-11 g/dl; and
the specified time frame is at least about 8 weeks.

10. The method of claim 1, wherein the patient's aligned hemoglobin time series is classified as “fluctuating” if either: the difference between the maximum hemoglobin value and the minimum hemoglobin value is larger than 1.75 g/dL, or a portion of time that a weekly hemoglobin rate of change exceeds 0.1 g/DL/week is larger than 60% for the aligned hemoglobin time series.

11. A method for generating an aligned hemoglobin time series for a patient from multiple measurement sources, the method comprising:
accessing hemoglobin measurements (Hgb) for the patient;
accessing hematocrit measurements (HCT) for the patient measured during treatments on the patient, and identifying the hematocrit measurements that correspond to a start (pre HCT) and end (post HCT) of the treatments;
converting the pre HCT and post HCT to hemoglobin values pre Hgb and post Hgb;
assembling a hemoglobin time series of the Hgb, pre Hgb, and post Hgb for the patient;
determining an adjustment to values of the pre Hgb and values of the post Hgb to improve alignment with values of the Hgb;
applying the adjustment to the values of the pre Hgb and the values of the post Hgb, yielding aligned pre Hgb values and aligned post Hgb values; and
populating the aligned hemoglobin time series for a desired time window using Hgb during treatment sessions when Hgb data is available within the desired time window, and using aligned pre Hgb values and aligned post Hgb values during treatment sessions when Hgb data is unavailable.

12. The method of claim 11, further comprising:
selectively filtering the hemoglobin measurements (Hgb), wherein the filtering excludes Hgb values that are outside a first threshold from a first moving average; or
selectively filtering the pre HCT and post HCT, wherein the filtering excludes hematocrit measurements that are outside a second threshold from a second moving average for the hematocrit measurements.

13. The method of claim 11, wherein:
determining the adjustment includes determining an offset correction by computing a mean offset between the pre Hgb values and the Hgb values, and subtracting the mean offset from the pre Hgb values and post Hgb values to yield offset pre Hgb values and offset post Hgb values.

14. The method of claim 13, wherein:
determining the adjustment includes determining an interpolation factor between offset pre Hgb values and offset post Hgb values such that the average deviation between a resulting interpolated hemoglobin and the Hgb is minimized.

15. The method of claim 14, wherein:
applying the adjustment to the pre Hgb values and post Hgb values includes subtracting the mean offset from the pre Hgb values and post Hgb values and applying the interpolation factor yielding the aligned pre Hgb values and aligned post Hgb values.

16. The method of claim 11, wherein:
the hemoglobin measurements (Hgb) are from laboratory hemoglobin measurements performed on blood samples of the patient; and
the hematocrit measurements (HCT) are measured by non-invasive photo-optical sensors during extracorporeal treatments on the patient.

17. The method of claim 11, wherein the hematocrit measurements that correspond to the end of a treatment that was ended unexpectedly are disregarded.

18. The method of claim 11, wherein the patient is a dialysis patient and the hematocrit measurements are collected as part of the patient's regular dialysis treatments, the method further comprising:
feeding the aligned hemoglobin time series into a predictive model to determine an erythropoiesis-stimulating agent (ESA) dose for the patient.

19. The method of claim 11, wherein the aligned hemoglobin time series spans a period of at least 90 days and includes at least 15 values.

20. A system comprising:
a processing circuit; and
memory having executable instructions stored thereon, which when executed by the processing circuit, causes the processing circuit to:
access patient parameters associated with the patient, wherein the patient parameters include an aligned hemoglobin time series for the patient generated from multiple measurement sources;
access a physiology-based model;
adapt the physiology-based model into a patient specific model that predicts future hemoglobin levels for the patient based on one or more erythropoiesis-stimulating agent (ESA) dosing regimens, wherein adapting the physiology-based model to the patient specific model utilizes the patient parameters, and generates estimates of patient-specific physiological characteristics;
run simulations with the patient specific model and determine a recommended ESA dose that the model predicts will cause one or both of the patient's hematocrit or hemoglobin concentration to reach a desired range within a specified time frame; and
provide the recommended ESA dose to the patient's healthcare provider.
